(19) United States
(12) Patent Application Publication
(10) Pub. No.: US 2024/0026267 A1
(43) Pub. Date: Jan. 25, 2024

(54) METHODS, SYSTEMS, AND DEVICES FOR SEPARATING AND CHARACTERIZING CIRCULATING RARE CELLS FROM BIOLOGICAL SAMPLES

(71) Applicant: University of Georgia Research Foundation, Inc., Athens, GA (US)

(72) Inventors: Leidong Mao, Watkinsville, GA (US); Yang Liu, Albany, CA (US); Wujun Zhao, Waunakee, WI (US)

(21) Appl. No.: 18/224,717

(22) Filed: Jul. 21, 2023

Related U.S. Application Data
(60) Provisional application No. 63/391,984, filed on Jul. 25, 2022.

(51) Int. Cl. C12M 3/06; C12M 1/12; C12M 1/42

(52) U.S. Cl. CPC C12M 23/16; C12M 25/16; C12M 35/06

(57) ABSTRACT

The present disclosure provides for microfluidic devices, systems, kits, and methods of using multi-stage microfluidic devices are provided for high throughput sorting, separation/enrichment of target rare cells from a sample, and can additionally provide for characterization/phenotyping of circulating tumor cells (CTCs) and other unlabeled rare cells in a biological sample such as blood, where the rare cells do not need to be labeled.

### CROSS-REFERENCE TO RELATED APPLICATIONS

This application claims the benefit of and priority to U.S. Provisional Application Ser. No. 63/391,984, having the title “METHODS, SYSTEMS, AND DEVICES FOR SEPARATING AND CHARACTERIZING CIRCULATING RARE CELLS FROM BIOLOGICAL SAMPLES,” filed on Jul. 25, 2022, the disclosure of which is incorporated herein in by reference in their entireties.

### STATEMENT REGARDING FEDERALLY SPONSORED RESEARCH OR DEVELOPMENT

This invention was made with government support under Grant Nos. 1150042, 1659525 and 1648035 awarded by the National Science Foundation, by No. UL1TR002378 and 1R41EB028191-01 awarded by the National Institutes of Health. The government has certain rights in the invention.

### BACKGROUND

Rapid separation of rare target cells in a large volume of biological samples provides unique opportunities for disease diagnostics and treatment. The ability to separate and enrich populations of scarce target cells, such as rare circulating tumor cells (CTCs), from biological samples can provide valuable research tools and insights into diseases such as metastatic cancers. The limited availability and difficulty of obtaining useful amounts of rare CTCs is a hurdle in such research. However, even with advanced technologies for cell separation, the limiting throughput, high cost and low separation resolution of currently available cell separation platforms still prevent the effectiveness of such technologies in processing large volumes of biological samples.

Profiling circulating tumor cells (CTCs) in cancer patients' blood samples is critical to understanding the complex and dynamic nature of metastasis. This task is further complicated by the fact that CTCs are not only extremely rare in circulation but also highly heterogenous in their molecular programs and cellular functions, even within a single sample. Label-based CTC separation technologies were developed to selectively enrich a subset of CTCs from blood, primarily through the use of specific biological markers including epithelial cell adhesion molecule (EpCAM). These antigen-based labels were a rate-limiting factor in effective CTC separation, as the inherent heterogeneity of CTCs render these technologies ineffective for general use. The vast array of various biomarkers that might or might not be expressed, and which cannot be predicted to remain expressed in CTCs undergoing Epithelial-to-Mesenchymal Transitions (EMT) are cumbersome and confounding in these label-based methods. Furthermore, most label-based technologies do not conveniently enable comprehensive molecular analysis of separated CTCs because they are either dead or immobilized to a surface. Thus, a variety of label-free methods have been developed to exploit specific physical markers in order to deplete non-CTCs in blood and thereby enrich cancer cells. While such methods may be used to separate CTCs based upon, for example, size, the existence of large white blood cells, such as monocytes, that may have overlapping sizes with CTCs complicate these label-free methods and reduce the purity of the sample obtained. Other devices have attempted to incorporate two or more of these methods, but still suffer from the time-consuming and laborious sample preparation due to the complications discussed above for labeling CTCs.

Furthermore, due to the heterogeneity of CTCs in the blood and the different health threat posed by different types of CTC's there is a great interest in further characterizing the types of CTCs circulating in a patient's bloodstream. For instance, only some CTC's transition to an invasive phenotype with the ability to actively invade distant organs. Identification of these invasive phenotype CTCs that pose the greatest threat of metastasis could help predict patient outcomes, identify those in need of more aggressive therapies, and identify new treatments. Thus, in addition to separation of CTC's from other components of blood, there is additional interest in further characterization of isolated CTC's with respect to their phenotype and to differentiate and identify passive and invasive CTC phenotypes.

There remains a need for improved devices and methods for separating circulating tumor cells that overcome the aforementioned deficiencies.

### SUMMARY

Embodiments of the present disclosure provides for microfluidic devices, systems, kits, and methods of using multi-stage microfluidic devices are provided for high throughput sorting, separation/enrichment of target rare cells from a sample, and can additionally provide for characterization/phenotyping of circulating tumor cells (CTCs) and other unlabeled rare cells in a biological sample such as blood, where the rare cells do not need to be labeled.

In an aspect the present disclosure provides for a method of enriching target rare cells in a biological sample, the method comprising: combining the biological sample with a plurality of magnetic microbeads adapted to specifically conjugate with white blood cells (WBCs) such that a majority of WBCs in the sample are conjugated to one or more magnetic microbeads to produce a magnetically labeled biological sample; combining the magnetically labeled biological sample with a colloidally stable ferrofluid to produce a mixed ferrofluid biological sample; flowing the mixed ferrofluid biological sample through an inertial focusing stage comprising two or more sigmoidal microchannels with a plurality of alternating curvatures, such that rare cells and WBCs in the mixed ferrofluid biological sample are focused into one or more or two or more narrow focused fluid sample streams; flowing the one or more or two or more focused fluid sample streams through a ferrohydrodynamic separation stage comprising a ferrohydrodynamic separation channel and a magnetic source configured to produce a substantially symmetric magnetic field having a field maximum along an inner longitudinal axis of the ferrohydrodynamic separation channel sufficient to cause the white blood cells conjugated to the magnetic beads flowing into the ferrohydrodynamic separation channel to be focused toward a central longitudinal axis of the ferrohydrodynamic separation channel and to cause target rare cells in the sample to be deflected towards an outer portion of the ferrohydrodynamic separation channel; separating the magnetic-bead-conjugated WBCs flowing along a central longitudinal axis of the ferrohydrodynamic separation channel from target rare cells flowing along an outer portion of the ferrohydrodynamic separation channel to produce an enriched biological sample. The rare cells can be circulating tumor cells (CTCs) and the biological sample is a red blood cell-lysed blood sample from a patient. The flow rate of the mixed ferrofluid biological sample can be about 500-2000 μl/min. The concentration of the ferrofluid can be about 0.005-0.05%. Over 95% of WBC's cab be separated from the biological sample to produce the enriched biological sample. In an aspect, over 99% of WBC's can be separated from the biological sample to produce the enriched biological sample. The magnetic source can comprise an array of magnets comprising a first array and second array, wherein the ferrohydrodynamic separation stage is sandwiched between and substantially centrally aligned between the first magnet array and the second magnet array, wherein the magnets in the first array are oriented to repel the magnets in the second array. The array of magnets comprises six magnets that can be arranged in a sextuple configuration. The method further comprises introducing the enriched biological sample into a microfluidic chemotactic cell migration unit and sorting the target rare cells into different migratory phenotypes based on at least one of a speed and distance of migration of target rare cells with respect to one or more chemo-modulatory compounds flowing in a portion of the microfluidic chemotactic cell migration unit. The target rare cells can be circulating tumor cells (CTCs) and the one or more chemo-modulatory compounds comprises at least one compound that is a chemoattractant for CTCs having an invasive phenotype. The chemoattractant for CTCs can be selected from the group consisting of Fetal Bovine Serum (FBS), epidermal growth factor (EGF), and basic fibroblast growth factor (bFGF). The rare cells can be target circulating tumor cells (CTCs) and the one or more chemo-modulatory compounds comprises at least one compound that is a migration inhibitor for WBCs. The at least one migration inhibitor compound for WBCs can be Slit2. The enriched biological sample can be incubated in microfluidic chemotactic cell migration unit with the chemo-modulatory compounds for about 3 to 48 hours. A gradient of chemo-modulatory compounds can be maintained by continuous perfusion during the incubation time. Conjugating the WBCs in the sample to magnetic microbeads comprises: combining the WBC with one or more WBC biomarker antibodies and then conjugating the biomarker antibodies to the magnetic microbeads. The biomarker antibodies can be biotinylated and the magnetic microbeads are coated with streptavidin, and wherein the one or more or two or more WBC biomarker antibodies are selected from the group consisting of: CD45, CD45RA, CD66b, CD16, and CD3.

An aspect of the present disclosure provides for an integrated inertial ferrohydrodynamic cell separation (i²FCS) microfluidic unit comprising: a sample inlet configured to receive a mixed ferrofluid biological sample comprising a biocompatible ferrofluid combined with a biological sample comprising target rare cells and magnetically labeled white blood cells (WBCs); a filter section fluidly connected to the sample inlet, the filter section comprising a first microfluidic channel and one or more filters configured to remove a first plurality of waste particles from the mixed ferrofluid biological sample; an inertial focusing stage fluidly connected to the filter section, wherein the first microfluidic channel splits into two or more sigmoidal microchannels with a plurality of alternating micro-curves, each sigmoidal microchannel configured to focus cells within the sample into a narrow stream to produce a focused fluid sample stream; a ferrohydrodynamic separation stage fluidly connected to the inertial focusing stage such that the focused fluid sample streams from the two or more sigmoidal microchannels flow into a ferrohydrodynamic separation channel; a waste outlet fluidly connected to and axially aligned with the ferrohydrodynamic separation channel to receive materials flowing through a central portion of the channel, one or more target cell outlets each fluidly connected to the ferrohydrodynamic separation channel and offset from the center of the channel and configured to receive material flowing near the outer portion of the channel; and one or more magnetic sources adjacent to ferrohydrodynamic separation stage and configured to produce a substantially symmetric magnetic field having a field maximum along a length of the ferrohydrodynamic separation channel sufficient to cause the white blood cells conjugated to the magnetic beads in the ferrohydrodynamic separation channel to be focused toward a center of the channel and to exit the channel via the waste outlet and to cause target rare cells to be deflected towards an outer portion of the ferrohydrodynamic separation channel and to exit the channel via the one or more target cell outlets. The magnetic source comprises an array of magnets comprising a first array and second array, wherein the ferrohydrodynamic separation stage is sandwiched between and substantially centrally aligned between the first magnet array and the second magnet array, wherein the magnets in the first array are oriented to repel the magnets in the second array. The array of magnets comprises six magnets arranged in a sextuple configuration. A distance between a center of the ferrohydrodynamic separation channel and a magnet junction of the array of magnets is from about 0-50 μm. The array of magnets produces a magnetic flux density of about 1.0-3.0 T within the ferrohydrodynamic separation channel and a magnetic flux gradient of about 500-5000 T m⁻¹. The number of alternating micro-curves in each of the sigmoidal microchannels comprises about 30-40 micro-curves. The alternating micro-curves comprise alternating small and large micro-curves. The diameter under each large micro-curve is about 600-2400 μm and the diameter under each small micro-curve is about 300-800 μm. An interior channel width of each serpentine focusing channel varies along the length of said channel, wherein the interior channel width at a crest portion of each smaller micro-curve is about 150-400 μm and wherein the interior channel width at a crest portion of each larger micro-curve is about 330-850 μm. The internal channel width of the ferrohydrodynamic separation channel is about 800-1600 μm. The ferrohydrodynamic separation channel can have a u-shaped curve, where the length of the channel before the curve is about 51000-59500 μm and the length after the curve is about 51500-60000 μm. The channel height in the inertial focusing stage and the ferrohydrodynamic separation phase is about 30-300 μm.

An aspect of the present disclosure provides for a combined integrated inertial ferrohydrodynamic cell separation (i²FCS) and cell migration device comprising: the i²FCS unit as described above and herein and a chemotactic cell migration unit comprising: an enriched sample inlet to receive an enriched biological sample from the i²FCS unit; a chemo-modulatory fluid inlet to receive a chemo-modulatory fluid comprising one or more chemo-modulatory compounds; one or more sample flow channels fluidly connected to the enriched sample inlet and configured to flow the enriched biological sample; one or more chemo-modulatory channels fluidly connected to the chemo-modulatory fluid inlet, configured to flow the chemo-modulatory fluid, and oriented substantially parallel to the one or more sample flow channels; a plurality of migration microchannels connecting at least one sample flow channel to at least one chemo-modulatory channels, each of the plurality of migration microchannels oriented substantially perpendicular to the sample flow channel and the chemo-modulatory channel and in fluidic communication with both the sample flow channel and the chemo-modulatory channel, wherein the height of each migration microchannel is smaller than the height of each of the sample flow channel and the chemo-modulatory channels; and at least one migratory target cell outlet at and end of a chemo-modulatory channel opposite the chemo-modulatory fluid inlet configured to collect migratory target cells that have migrated from the enriched sample inlet. Each migration microchannel can have dimensions to allow migration of a single cell at a time. Each migration microchannel has a width of about 7-50 μm, a height of about 3-8 μm, and a length of about 200-2000 μm.

An aspect of the present disclosure provides for a cell separation system for enriching target rare cells in a biological sample and characterizing the target rare cells, the system comprising: a plurality of magnetic microbeads adapted for specific conjugation to white blood cells in the biological sample to provide a magnetically labeled biological sample having a majority of the white blood cells in the biological sample conjugated to two or more magnetic microbeads; a biocompatible ferrofluid comprising a plurality of magnetic nanoparticles and a biocompatible surfactant, the biocompatible ferrofluid adapted to be combined with the magnetically labeled biological sample and an optional biocompatible carrier fluid to make a mixed ferrofluid biological sample; the integrated i²FCS microfluidic unit as described above and herein; one or more chemo-modulatory compounds that affect the migratory behavior of one or more different phenotypes of target cells; a chemotactic cell migration unit comprising: an enriched sample inlet to receive an enriched biological sample from the i²FCS unit; a chemo-modulatory fluid inlet to receive a chemo-modulatory fluid comprising the one or more chemo-modulatory compounds; one or more sample flow channels fluidly connected to the enriched sample inlet and configured to flow the enriched biological sample; one or more chemo-modulatory channels fluidly connected to the chemo-modulatory fluid inlet, configured to flow the chemo-modulatory fluid, and oriented substantially parallel to the one or more sample flow channels; a plurality of microchannels connecting at least one sample flow channel to at least one chemo-modulatory channels, each of the plurality of microchannels oriented substantially perpendicular to the sample flow channel and the chemo-modulatory channel and in fluidic communication with both the sample flow channel and the chemo-modulatory channel, wherein the height of each microchannel is smaller than the height (or width) of each of the sample flow channel and the chemo-modulatory channels; and at least one migratory target cell outlet at and end of a chemo-modulatory channel opposite the chemo-modulatory fluid inlet configured to collect migratory target cells that have migrated from the enriched sample inlet.

### BRIEF DESCRIPTION OF THE DRAWINGS

Further aspects of the present disclosure will be readily appreciated upon review of the detailed description of its various embodiments, described below, when taken in conjunction with the accompanying drawings.

FIGS. 1A-1B are schematic illustrations comparing the separation mechanism of integrated inertial ferrohydrodynamic cell separation with magnetically labeled WBC (i²FCS) (FIG. 1A) and size-based inertial-ferrohydrodynamic cell separation (iFCS) (FIG. 1B).

FIG. 2 is a schematic illustration of an embodiment of a system of the present disclosure showing the integrated inertial ferrohydrodynamic separation unit (i²FCS) and the cell migration unit.

FIG. 3A is a schematic of an embodiment of an integrated inertial ferrohydrodynamic separation (i²FCS) unit of the present disclosure, showing the filter stage, the inertial focusing stage, and the magnetic separation stage. The schematic illustration in FIG. 3B shows the arrangement of the magnet(s) in the embodiment of the inertial ferrohydrodynamic separation unit of FIG. 3A, with the insert showing a close up of the arrangement of the magnet with respect to the magnetic separation channels and the magnet junction. FIG. 3C illustrates a close-up view showing the configuration and the dimensions of an embodiment of the inertial focusing stage, and FIG. 3D illustrates a close-up view of an embodiment of the outlet from the magnetic separation stage showing geometry and dimensions of an embodiment of the outlet to optimize recovery of CTCs and removal of WBCs.

FIG. 4 illustrates an overview of the integrated inertial ferrohydrodynamic cell separation (i²FCS) scheme and embodiment of the inertial ferrohydrodynamic separation unit. (a) Two stages (inertial focusing and ferrohydrodynamic separation) are integrated into the i²FCS device. A first inertial focusing stage focuses suspended cells in the ferrofluid into a narrow stream in sigmoidal microchannels with alternating curvatures. A second ferrohydrodynamic separation stage directs unlabeled CTCs toward the upper and lower locations of the channel, and WBCs toward the center of the channel, using a symmetric magnetic field distribution with its maximum aligned to the channel center from a sextuple magnet array. Arrows indicate the gradient of the magnetic field. (b) Schematic illustration of circulating tumor cells (CTCs) experiencing “diamagnetophoresis” and labeled white blood cells (WBCs) experiencing both “diamagnetophoresis” and “magnetophoresis” in a colloidal magnetic nanoparticle suspension (ferrofluid). Unlabeled CTCs with close to zero magnetization experience magnetic buoyancy under an external magnetic field, which is generated by the magnetic nanoparticle-induced pressure imbalance on the cell's surface, and is proportional to the cell's volume. This force moves the CTCs toward the minimum of a non-uniform magnetic field. Magnetic bead labeled WBCs experience both “diamagnetophoresis” from its cell surface and “magnetophoresis” from its attached beads in a ferrofluid and move towards the maximum of the magnetic field because magnetophoretic effect outweighs diamagnetophoretic effect. Color bar indicates the relative amplitude of the magnetic field. Red arrows show the direction of cell movement, small black arrows on the cell surface show the direction of magnetic nanoparticle induced surface pressure on cells. (c) Top view of the i²FCS device. Cells are injected into the sample inlet and purified with the debris filters to remove debris larger than 60 μm. After inertially focused in the inertial focusing stage, cells of different magnetization were separated in the ferrohydrodynamic separation stage. (d) A photo of the i²FCS device microchannel (left) and assembled i²FCS device with sextuple magnets inside an aluminum holder (right). (e) Images of i²FCS device in operation. Red (15 μm diameter) diamagnetic polystyrene beads and green (11.8 μm diameter) magnetic polystyrene beads were mixed in a ferrofluid and injected into the device for imaging: (1) particles in the debris filter; (2) particles prior to inertial focusing stage; (3) particles after inertial focusing; (4-5) particles in the ferrohydrodynamic separation stage. Sample flow rate in this experiment was 500 μL min⁻¹ (30 mL h⁻¹).

FIG. 5 illustrates optimization of the magnetic field to achieve high magnetic flux density and flux density gradient in an embodiment of an i²FCS device/system of the present disclosure. Six neodymium permanent magnets (each with a dimension of L×W×H, 50.8×6.35×6.35 mm) were arranged in a sextuple configuration in the i²FCS device. (a) Distribution of the magnetic flux density of the sextuple magnets array in the y-z plane (x=0). Microfluidic channels were placed between the junctions of magnets. (b) A symmetric magnetic field was generated in the i²FCS device channel with the highest magnetic flux density located at the center of the microchannel in the y-z plane (x=0). (c) Distribution of the magnetic flux density of the magnets array in the x-y plane (z=0). Microchannels (cyan dashed lines) were placed in the area with the highest magnetic flux density in a symmetric pattern. (d) Diamagnetic CTCs (green) moved to the area with a minimal magnetic field while magnetically labeled WBC (gray) moved to the area with a maximal magnetic field in the i²FCS device channel. (e) Distributions of magnetic flux density gradient in the microchannel. A magnetic flux density gradient of 670 T m⁻¹ in the y-z plane (x=0) was located near the edge of the channel.

FIG. 6 illustrates system optimization of embodiments of i²FCS devices for the isolation of CTCs (down to 10 cells per mL) with high recovery rate, low WBC contamination, and ultrahigh throughput. (a) Optimization of magnetic beads labeling of WBCs. Left: distribution of the number of magnetic beads labeled on WBCs (n=2000). On average, there are 21±9 (mean±s.d.) Dynabeads conjugated onto a single WBC. More than 99.95% of WBCs are labeled with at least two beads. Inset is a WBC labeled with multiple Dynabeads. Right: distribution of magnetic content in labeled WBCs. More than 99.9% of WBCs has a volume fraction of magnetic content larger than 0.015% (v/v). (b) Effective diameter distribution of human breast cancer cell line (MCF7). Its diameter is 20.6±6.6 μm (mean±s.d., n=5000). (c-d) Optimization of ferrofluid concentration and flow rate for i²FCS to achieve maximal separation distance (ΔY) between CTCs and WBCs. The optimized ferrofluid concentration is 0.015%, while the flow rate is 1000 μL min⁻¹ (60 mL h⁻¹). During the optimization, the magnetic flux density and its gradient are the same as in FIG. 5. (e) Simulation of cell distributions at the cross-section of the outlet of an i²FCS device/unit. 10,000 MCF7 cells and 10,000 labeled WBCs were used to mimic the heterogeneity of blood sample. After device processing, 100% of CTCs are collected from CTCs outlets with (5 of 10,000) WBCs contamination. 99.95% of WBCs are depleted from WBCs outlet. MCF7 cancer cells are represented with red dots and WBCs are represented with white dots. The color of the edges of the dots represents the density of the cells at that location. (f-g) Quantification of CTC and WBC distribution at the end of the i²FCS device. (h) Dependence of final position (Y) of WBCs along the channel width at the outlet of the device on the magnetic content of individual WBCs. Simulation parameters of e-h include ferrofluid with a concentration of 0.015% and a sample flow rate of 1000 μL min⁻¹ (60 mL h⁻¹). (i) Visualization of cancer cell isolation and WBC depletion (left: epifluorescence; right: bright field). Diamagnetic cancer cells were collected from upper and bottom outlets while the WBCs labeled with magnetic beads were depleted from the middle outlet. The cells were suspended in 0.015% ferrofluid and processed with a flow rate of 1,000 μL min⁻¹ (60 mL h⁻¹).

FIG. 7 illustrates characterization of performance of an embodiment of an i²FCS device/unit using cancer cell lines spiked into blood from healthy donors. Cancer cells and WBCs were processed in 0.015% (v/v) ferrofluid with a flow rate of 1000 μL min⁻¹ (60 mL h⁻¹) to achieve high cancer cell recovery rate and low WBC contamination. (a) Spike-in experiments indicated a high recovery rate (100%) of cancer cells. Experiments with different number (10, 50, 100, and 200) of MCF7 cells spiked into 1 mL of labeled WBCs. An average recovery rate of 100% was achieved (R²=1, n=3). (b) Diameter distribution of 11 cancer cell lines. (c) Recovery rates of spiked cancer cells (˜100 cells per mL, total: 15 mL). The recovery rates of 100.00±0.00%, 99.33±0.49%, 99.67±0.47%, 99.83±0.24%, 99.67±0.47%, 99.67±0.42%, 100±0.00%, 100±0.00%, 100±0.00%, 99.67±0.94%, and 98.83±1.03% were achieved for MCF7 (breast cancer), MDA-MB-231 (breast cancer), HCC1806 (breast cancer), HCC70 (breast cancer), A549 (non-small cell lung cancer), H1299 (non-small cell lung cancer), H3122 (non-small cell lung cancer), H520 (non-small cell lung cancer), DMS79 (small cell lung cancer), H69 (small cell lung cancer), and PC-3 (prostate cancer) cell lines, respectively (mean±s.d., n=3). (d) The corresponding WBC contaminations for each cell line are 547±113, 487±100, 553±80, 630±147, 473±80, 507±153, 467±82, 440±73, 480±87, 453±93, and 540±73 WBCs per mL blood (mean±s.d., n=3). (e) Short term viability of H1299 lung cancer cells before and after i²FCS processing was determined to be 99.31±0.42% (mean±s.d., n=3) and 98.10±1.35% (mean±s.d., n=3), respectively. (f) Images of Live/Dead staining before and after enrichment, and long-term (48 hours) proliferation test. Cells were stained with Calcein AM (green, live cells) and EhD-1 (red, dead cells). (g) Immunofluorescence images of H1299 lung cancer cell and WBC contamination after enrichment. Five channels including EpCAM (green), CD45 (red), N-cadherin (N-Cad, cyan), Vimentin (Vim, magenta), and DAPI (blue) were used.

FIG. 8 illustrates biochemical phenotyping of CTCs isolated from two metastatic lung cancer patients (n=2). (a) Immunofluorescence images of 11 selected CTCs and 1 WBCs from the two patients. Five channels were used in the immunofluorescent staining including epithelial CTC marker EpCAM (green), leukocyte marker CD45 (red), mesenchymal CTC markers N-cadherin (N-Cad, cyan) and vimentin (Vim, magenta), and nucleus marker DAPI (blue). Cells with either epithelial positive (EpCAM+/CD45−/DAPI+), mesenchymal positive (N-cad+/CD45−/DAPI, Vim+/CD45−/DAPI+, and N-cad+/Vim+/CD45−/DAPI+), or mixed epithelial and mesenchymal positive (EpCAM+/N-Cad+/Vim+/CD45−/DAPI). (b) Statistic analysis of cell diameters of collected CTCs from the patients. CTCs from patient A (lung cancer, stage IV) had a diameter of 13.29±6.13 μm (mean±s.d., n=75); CTCs from patient B (lung cancer, stage IV) had a diameter of 10.22±4.85 μm (mean±s.d., n=70). (c) Quantitative analysis of surface antigens expression of individual CTCs from the patients. Epithelial positive CTCs (E) were identified as EpCAM+/Vim−/N-cad-. Mixed epithelial and mesenchymal CTCs (E/M) were identified as EpCAM+/Vim+/N-cad− and EpCAM+/Vim+/N-cad+. Mesenchymal CTCs (M) was identified as EpCAM−/Vim+/N-cad+, EpCAM−/Vim+/N-cad−, and EpCAM−/Vim−/N-cad+.

FIG. 9 illustrates functional phenotyping of CTCs isolated from one metastatic lung cancer patient (n=1) in an embodiment of a system of the present disclosure having a chemotactic cell migration unit. (a) Illustration of the procedure for CTC isolation via an i²FCS device, and migration characterization of isolated CTCs via a single-cell migration device. 20 mL of patient blood was processed using an i²FCS device. A quarter of isolated cells were used in the single cell migration microfluidic device. (b) Cells were loaded into the top microchannel at the beginning of the migration assay. A gradient of growth factors was established over a 24-hour period via continuous perfusion to allow phenotyping of cells with different migratory distance and speed. The direction of the arrow indicates the gradient of the growth factors. (c) Characterization of the migration device using H1299 lung cancer cells. Bright field images of migrated H1299 cells at the end of a 24-hour period shows migratory versus non-migratory cell populations. (d) Migration distance of CTCs (n=207) from Patient B's blood sample. Cells were loaded into the migration device at a similar starting position (dashed line, Y=200 μm). A gradient of growth factors (10% fetal bovine serum, FBS; epidermal growth factor, EGF; and basic fibroblast growth factor, bFGF) was used to guide the migration of CTCs, while a gradient of chemorepellent (Slit2) were used to inhibit the migration of carryover WBCs. After the 24-hour migration, cells in the device were immunofluorescently stained with anti-EpCAM (green), anti-CD45 (red), anti-vimentin (white), and DAPI (green) to identify the cell types. CTCs was identified as EpCAM+/CD45−/DAPI+, EpCAM+/Vim+/CD45−/DAPI+, and Vim+/CD45−/DAPI+, while WBCs was identified as EpCAM−/Vim−/CD45+/DAPI+. (e) Migration speed of CTCs from Patient B was 0.26±0.19 μm min⁻¹ (mean±s.d., n=207). (f) Bright field and immunofluorescence images of one non-migratory CTC (left) and one migratory CTC (right) of patient B, with their final migration position circled in figure (d). (g) Immunofluorescent images of low migratory cells (left, found in the loading channel of the migration device) and high migratory cells (right, found in the migration tracks of the device).

FIG. 10 illustrates Table 1, which is a comparison of design, operation and performance of CTC isolation between CTC-iChip and i²FCS.

### DETAILED DESCRIPTION

In various aspects, microfluidic systems, devices, and methods of using microfluidic devices are provided for separating/enriching rare cells, such as circulating tumor cells (CTCs), in a biological sample such as whole blood. The methods do not involve labeling of the rare cells and are capable of high throughputs with high levels of retention and separation of the rare cells. In embodiments, the methods, systems, and devices also provide for phenotypically characterizing the separated/enriched rare cells according to certain migration phenotypes.

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting. The skilled artisan will recognize many variants and adaptations of the embodiments described herein. These variants and adaptations are intended to be included in the teachings of this disclosure and to be encompassed by the claims herein.

Embodiments of the present disclosure will employ, unless otherwise indicated, techniques of biochemistry, molecular biology, microfluidics, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Although any methods and materials similar or equivalent to those described herein can also be used in the practice or testing of the present disclosure, the preferred methods and materials are now described. Functions or constructions well-known in the art may not be described in detail for brevity and/or clarity.

It should be noted that ratios, concentrations, amounts, and other numerical data can be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to about 5%” should be interpreted to include not only the explicitly recited values of about 0.1% to about 5%, but also include individual values (e.g., 1%, 2%, 3%, and 4%) and the sub-ranges (e.g., 0.5%, 1.1%, 2.2%, 3.3%, and 4.4%) within the indicated range. Where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In some embodiments, the term “about” can include traditional rounding according to significant figures of the numerical value. In addition, the phrase “about ‘x’ to ‘y’”, where ‘x’ and ‘y’ are numerical values, includes “about ‘x’ to about ‘y’”.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

### Definitions

Unless defined otherwise, all technical and scientific terms used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

The articles “a” and “an,” as used herein, mean one or more when applied to any feature in embodiments of the present invention described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used.

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

In this disclosure, “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, “about,” “approximately,” “substantially,” and the like, when used in connection with a numerical variable, can generally refers to the value of the variable and to all values of the variable that are within the experimental error (e.g., within the 95% confidence interval for the mean) or within +/−10% of the indicated value, whichever is greater. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” indicates that the subsequently described event or circumstance can or cannot occur, and that the description includes instances where said event or circumstance occurs and instances where it does not.

As used herein, “kit” refers to a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” refers to documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, “attached” can refer to covalent or non-covalent interaction between two or more molecules. Non-covalent interactions can include ionic bonds, electrostatic interactions, van der Walls forces, dipole-dipole interactions, dipole-induced-dipole interactions, London dispersion forces, hydrogen bonding, halogen bonding, electromagnetic interactions, 7-7 interactions, cation-7 interactions, anion-7 interactions, polar 7-interactions, and hydrophobic effects.

A “biocompatible” substance or fluid, as described herein, indicates that the substance or fluid does not adversely affect the short-term viability or long-term proliferation of a target cell within a particular time range.

“Curved” or “curve,” as described herein, indicates a non-linear shape, where curved can include a single curve, multiple curves, and multi-directional curves, including crescent-shaped, U-shaped, serpentine, sigmoidal, and the like.

As used herein, the term “cells/particles” refers to particles, cells, or a combination of both, in a sample or mixture.

As used herein the term “chemogradient” refers to a fluid chemical gradient created in some embodiments of the present disclosure characterized by a varying % concentration of a chemical compound (e.g., a chemo-modulatory compound) in one area of the device of the present disclosure to another. For instance, near where a chemo-modulatory fluid is introduced to the device the concentration of the chemo-modulatory fluid would be higher than in an area of the device further away from where the chemo-modulatory fluid was introduced.

The term “chemotactic” and “chemo-modulatory” as used herein refer to a chemical compound (e.g., a chemical compound in a fluid) that has the ability to induce chemotaxis, e.g., modulate the movement of a cell within a fluid, for instance, either toward (attraction) or away from (repellant) the chemical compound. For instance, a chemo-attractant has the effect of attracting a certain cell or cell-type, while a chemo-repellent has the effect of repelling a certain cell or cell-type. In embodiments, a chemo-modulatory fluid can include one or more fluids, e.g., a compound that may be a chemo-attractant for a first type of cell and a different compound that may be a chemo-repellent for a different type of cell. A chemo-modulatory compound or substance can induce chemotactic movement, chemotaxis, of a cell or entity in response to the chemical stimulus produced by the compound or substance.

As used herein, channels that are “substantially parallel” refers to channels of a device of the present disclosure that, with respect to a reference channel, while not absolutely parallel throughout the entire device, are parallel for a majority of the length of the channel, particularly in a portion of the channel in which cell migration is being observed.

Microfluidic Devices & Systems and Methods of Use Thereof

In various aspects, microfluidic devices, systems, kits, and methods of using multi-stage microfluidic devices are provided for high throughput sorting, separation/enrichment of target rare cells from a sample, and can additionally provide for characterization/phenotyping of circulating tumor cells (CTCs) and other unlabeled rare cells in a biological sample such as blood, where the rare cells do not need to be labeled.

Although microfluidics-based methods have been explored as a new avenue to enrich and study CTCs for the past decade, previous approaches were based on the use of specific tumor antigens (marker-dependent) or cell size threshold (cell size-dependent) for enrichment, and suffered disadvantages due to limitations of these approaches. For example, marker-dependent methods that relied on EpCAM or other combination of tumor cell surface antigens were rendered ineffective due to inherent heterogeneity of tumor subtypes. CTCs are highly heterogeneous in their biological and biophysical characteristics with multiple phenotypes co-existing, which can evolve dynamically over the course of metastasis. The significant difference among various markers and their expression levels in CTCs undergoing EMT was difficult to predict, resulting in incomplete recovery of CTCs from clinical samples.

Cell size-dependent methods, on the other hand, based on a presumed size difference between blood and cancer cells, proved largely ineffective because a significant percentage of CTCs in circulation were comparable or smaller than blood cells. Measurements of white blood cells (WBCs) and cultured cancer cells revealed that there was a significant size overlap between the two, and an appreciable percentage (e.g., ˜35% for DMS 79 and H69 small cell lung cancer cell lines) of cancer cells were smaller than ˜10 μm. In addition, clinically isolated CTCs were reported to be as small as ˜6 μm. As a result, few cell size-dependent methods could achieve complete recovery and low WBC contamination simultaneously. Furthermore, due to their fragile nature, CTCs need to be processed with gentle enrichment conditions to keep their viability and tumorigenic capability for downstream studies. As a result, current microfluidic methods for invasiveness phenotyping of tumor cells were mostly confined to cultured cancer cells rather than patient-derived CTCs. Thus, new devices and methods are needed that can enrich viable CTCs from patient samples, regardless of their surface antigens and size profiles, and maintain their functionality so that the properties of the invasive cells can be identified.

### Methods for Separation, Enrichment, and Characterization of Rare Cells in a Biological Sample

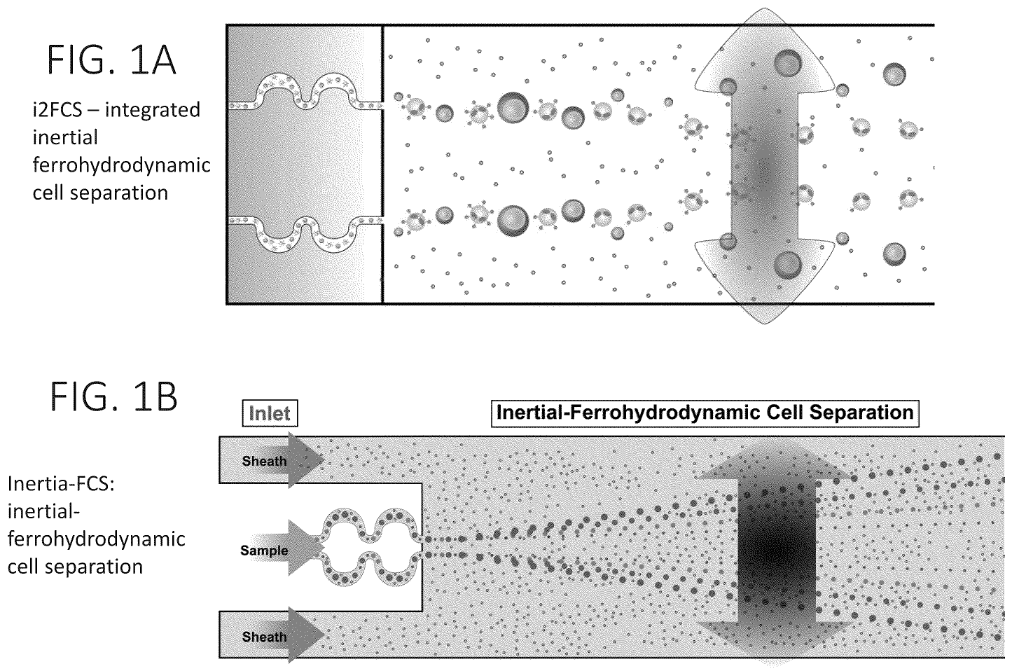

Broadly described, the present disclosure provides methods for separating/enriching rare cells from a sample without labeling the rare cells in the sample prior to separation and for further characterizing the phenotype (such as the migratory phenotype) of the separated/enriched rare cells as well as systems and devices for carrying out the methods of the present disclosure. FIGS. 1A and 1B illustrate the differences between earlier inertial ferrohydrodynamic cell separation (iFCS) techniques and the integrated inertial ferrohydrodynamic cell separation (i²FCS) techniques of the methods and systems of the present disclosure. Separation using iFCS used a combination of sheath flow and ferrohydrodynamic properties in a magnetic field to separate target cells from white blood cells purely as a function of size (FIG. 1B). On the other hand i²FCS involves magnetically labeling the white blood cells in the sample (much simpler and more efficient than labeling heterogeneous populations of rare cells in a sample), and using a combination of inertial focusing to focus the sample streams upon entry to the ferrohydrodynamic separation stage, where the magnetically labeled white cells are separated from unlabeled rare cells as a function of an applied magnetic field and the differing ferrohydrodynamic properties of the labeled WBCs and unlabeled rare cells. (FIG. 1A)

In general, methods of the present disclosure include providing a sample containing unlabeled rare cells (such as CTCs), magnetically labeling white blood cells in the sample, passing the sample through an inertial focusing device to produce a focused sample in a narrow stream, passing the focused sample through a ferrohydrodynamic separation device to separate magnetically labeled white blood cells in the sample from rare cells in the sample to produce an enriched sample having a higher concentration of target rare cells and a lower concentration of white blood cells than the original sample, and then passing the enriched sample through a chemotactic cell migration unit to further identify and characterize the target rare cells in the sample according to migratory phenotype in response to one or more chemo-modulatory/chemotactic agents. The present disclosure further provides variations of this method as well as various embodiments of systems and devices designed to carry out the methods of the present disclosure.

In embodiments, methods of enriching target rare cells in a biological sample include combining the biological sample with a plurality of magnetic microbeads adapted to specifically conjugate with white blood cells (WBCs) such that a majority of the WBCs are conjugated to one or more magnetic microbeads to produce a magnetically labeled biological sample. In embodiments, nearly all WBCs are conjugated to at least one magnetic microbead and a majority of WBCs in the sample are conjugated to two or more magnetic microbeads. In embodiments, conjugating the WBCs in the sample to magnetic microbeads includes combining the WBC with two or more WBC biomarker antibodies and then conjugating the biomarker antibodies to the magnetic microbeads. In embodiments, the biomarker antibodies are biotinylated and the magnetic microbeads are coated with streptavidin and multiple biomarker antibodies are used to increase the percent of magnetic microbeads that are conjugated to WBC and reduce the amount of unconjugated magnetic microbeads which can clog the channels of the microfluidic devices. In embodiments, the one or more or two or more WBC biomarker antibodies are selected from the group including but not limited to: CD45, CD45RA, CD66b, CD16, and CD3.

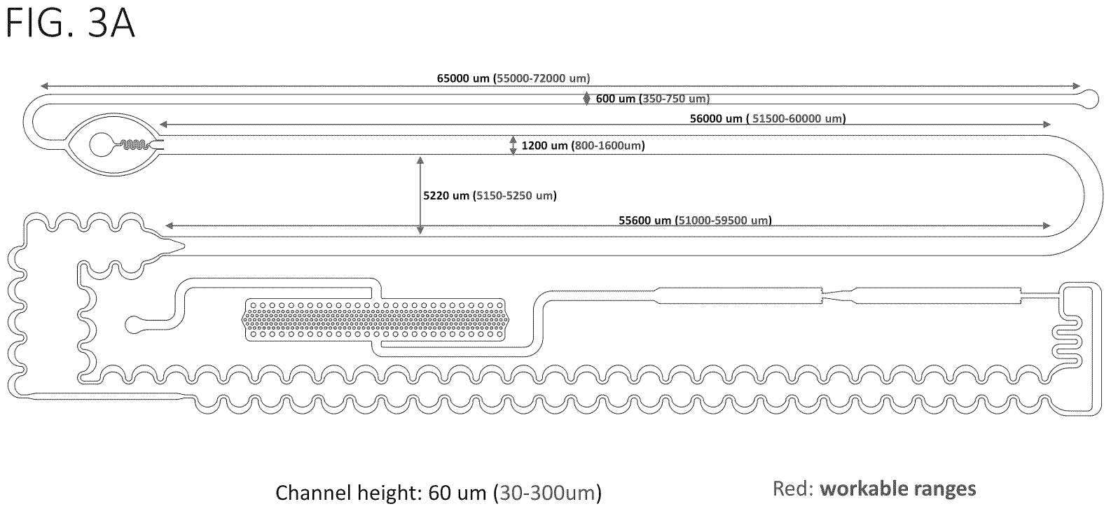

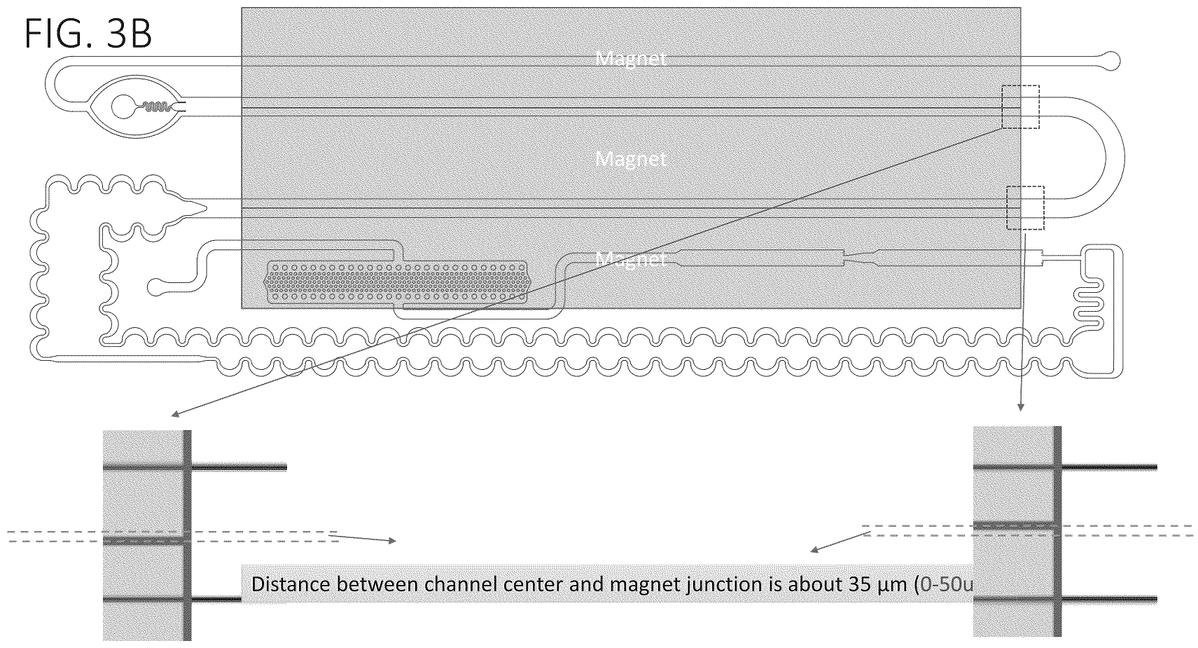

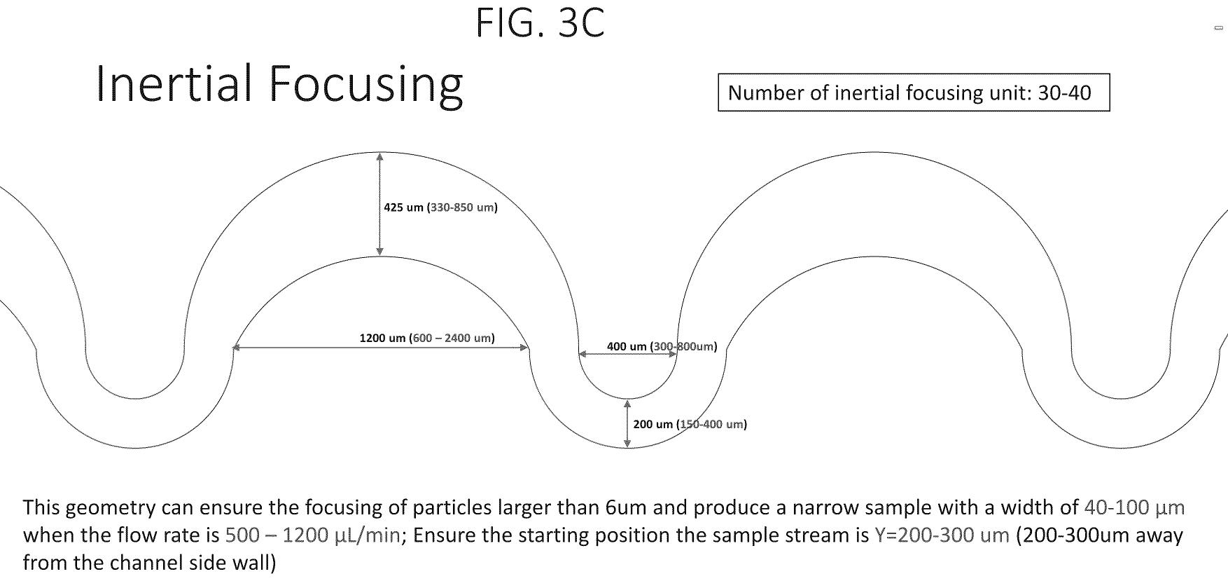

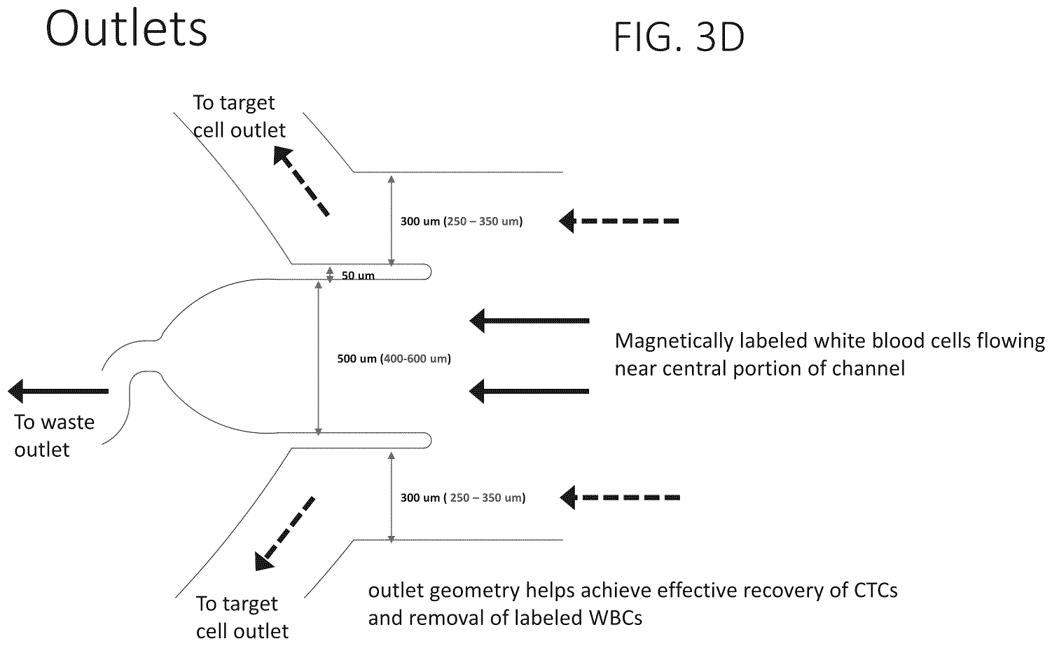

Then the magnetically labeled biological sample is combined with a colloidally stable ferrofluid to produce a mixed ferrofluid biological sample. The method then includes flowing the mixed ferrofluid biological sample through an inertial focusing stage. The inertial focusing stage includes two or more sigmoidal microchannels with a plurality of alternating curvatures (described further below, and an embodiment of which is depicted in FIG. 3C). The mixed ferrofluid biological sample is flowed through the inertial focusing stage at a flow rate such that rare cells and WBCs in the mixed ferrofluid biological sample are focused into one or more or two or more narrow focused fluid sample streams (see FIG. 1A). The two or more focused fluid sample streams are then flowed through a ferrohydrodynamic separation stage. The ferrohydrodynamic separation stage includes a ferrohydrodynamic separation channel and a magnetic source configured to produce a substantially symmetric magnetic field having a field maximum along an inner longitudinal axis of the ferrohydrodynamic separation channel sufficient to cause the magnetic-bead-conjugated white blood cells conjugated to the magnetic beads flowing into the ferrohydrodynamic separation channel to be focused toward a central longitudinal axis of the ferrohydrodynamic separation channel and to cause target rare cells in the sample to be deflected towards an outer portion of the ferrohydrodynamic separation channel (see FIG. 1A). Then the magnetic-bead-conjugated WBCs flowing along a central longitudinal axis of the ferrohydrodynamic separation channel are separated from target rare cells flowing along an outer portion of the ferrohydrodynamic separation channel to produce an enriched biological sample. In embodiments, the WBCs flowing along the central portion of the channel are separated by flowing into a waste outlet configured to capture particles flowing along a central longitudinal axis of the channel. In embodiments, the target rare cells flowing along outer portions of the ferrohydrodynamic separation channels (e.g., along the periphery of the channel) are collected in one or more cell collection outlets offset from the center of the channel and configured to receive material flowing near the outer portion of the channel.

In embodiments of the methods of the present disclosure the rare cells are circulating tumor cells (CTCs) and the biological sample is a red blood cell-lysed blood sample from a patient. The flow rate of the sample helps to optimize the inertial focusing as well as the ferrohydrodynamic separation and to achieve high throughput. In embodiments the flow rate of the mixed ferrofluid biological sample is about 500-2000 μl/min, such as about 500-1200 μl/min. The concentration of the ferrofluid also has an effect on the performance of the device, and affect the maximal separation distance between particles. In embodiments, the concentration of the ferrofluid is about 0.005-0.05%, such as about 0.015-0.03% (% v concentration of ferrofluid after mixing with the sample). As demonstrated in the examples below, methods of the present disclosure achieve separation of over 95% of WBC's from the biological sample to produce the enriched biological sample. In embodiments of these methods of the present disclosure, over 99% of WBC's are separated from the biological sample to produce the enriched biological sample, such as up to 99.992%.

In the ferrohydrodynamic separation stage, in embodiments, the magnetic source is an array of magnets comprising a first array and second array. In embodiments, the ferrohydrodynamic separation stage can be arranged between and substantially centrally aligned between the first magnet array and the second magnet array, such that the magnets in the first array are oriented to repel the magnets in the second array. In some embodiments, described in greater detail below, the array of magnets comprises six magnets arranged in a sextuple configuration. Greater details about the magnetic field properties are provided in the description below of the systems and devices of the present disclosure.

Methods of the present disclosure can also include further processing the enriched sample to further characterize the rare target cells enriched after the integrated inertial and ferrohydrodynamic separation. In embodiments, methods of characterizing the enriched target cells (e.g., CTCs) include observing certain migratory phenotypes of the target rare cells to differentiate cells with various migratory phenotypes. Embodiments of such methods include introducing the enriched biological sample into a microfluidic chemotactic cell migration unit and sorting the target rare cells into different migratory phenotypes based on at least one of a speed and distance of migration of target rare cells with respect to one or more chemo-modulatory compounds flowing in a portion of the microfluidic chemotactic cell migration unit. In some embodiments of the methods, the target rare cells are circulating tumor cells (CTCs) and the one or more chemo-modulatory compounds includes at least one compound that is a chemoattractant for CTCs having an invasive phenotype. In some embodiments the chemoattractant for CTCs is selected from, but not limited to factors such as, Fetal Bovine Serum (FBS), epidermal growth factor (EGF), and basic fibroblast growth factor (bFGF). In some embodiments, the rare cells are target circulating tumor cells (CTCs) and the one or more chemo-modulatory compounds comprises at least one compound that is a migration inhibitor for WBCs, such as but not limited to Slit2. In yet other embodiments, the chemo-modulatory compounds can include both a chemoattractant for CTCs or certain subtypes of CTCs as well as a compound that repels WBCs, such that CTCs are attracted to certain areas of the migratory separation stage that include the chemoattractant(s) and WBCs are repelled from certain regions of the device including the WBC repellant compound. In embodiments, the enriched biological sample is incubated in microfluidic chemotactic cell migration unit with the chemo-modulatory compounds for a sufficient period of time to allow for migration, such as from about 3 to 48 hours. For instance, the enriched biological sample can be incubated with the chemo-modulatory compounds for about 24 hours. In embodiments, a gradient of chemo-modulatory compounds is maintained by continuous perfusion during the incubation time.

Many other variations to the methods of the present disclosure can be envisioned by a skilled artisan, and some such variations are described in greater detail in the examples below.

### High Throughput Cell Separation, Enrichment, and Characterization Systems and Devices

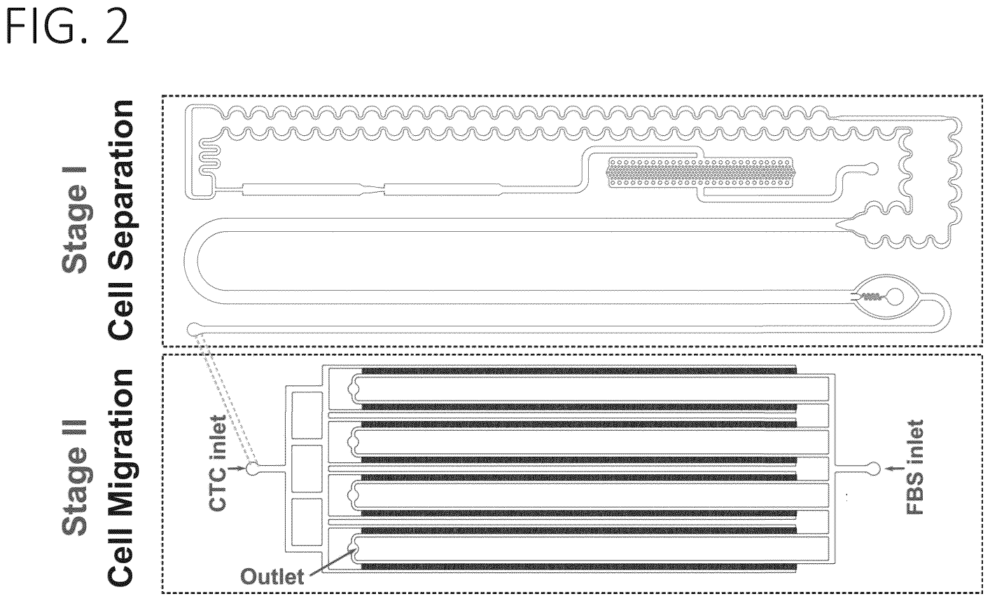

In some aspects, multi-unit, multi-stage microfluidic systems are provided for enriching and characterizing CTCs in a biological sample. In embodiments, the systems use a combination of a separation unit to separate target cells from non-target cells in a sample to produce an enriched sample and a migration unit to further characterize the target cells with respect to their migratory phenotype in response to different stimuli. In embodiments, systems of the present disclosure include an integrated inertial ferrohydrodynamics cell separation (i²FCS) approach to produce an enriched sample and a chemotactic cell migration approach for phenotypic characterization of the enriched cells. An illustration of a combined system of the present disclosure is shown in FIG. 2 of the present disclosure. In embodiments the systems of the present disclosure can include just an integrated inertial ferrohydrodynamic cell separation unit or can include both an integrated inertial ferrohydrodynamic cell separation unit and a chemotactic cell migration unit. In embodiments of rare cell separation and characterization systems, both the cell separation unit and the cell migration units can be integrated into a single device, while in other embodiments, the units by be in separate devices that together form a system of the present disclosure. Each unit will be described herein with respect to uses and variations, but it should be understood that the various embodiments of separation units and migration units can be used in varying combinations and configurations within the scope of the present disclosure.

### Magnetic Microbeads and Biocompatible Ferrofluids

Embodiments of the cell separation systems and kits of the present disclosure include a cell separation system and/or kit for enriching and/or characterizing CTCs in a biological sample. In addition to the microfluidic devices described herein, the cell separation/characterization systems of the present disclosure also include various compositions and compounds employed in the use of the systems and methods of the present disclosure. In embodiments, the cell separation systems can include a plurality of magnetic micro beads adapted for specific conjugation to white blood cells in the biological sample (e.g., the micro beads will not conjugate target cells, such as CTCs), a biocompatible ferrofluid including a plurality of magnetic nanoparticles and a biocompatible surfactant. The biocompatible ferrofluid can also be combined with the magnetically labeled biological sample and an optional biocompatible carrier fluid to make a mixed ferrofluid biological sample for processing in the systems/devices of the present disclosure. The systems/devices of the present disclosure allow enrichment of CTCs are size independent and do not require labeling the CTCs.

In embodiments the plurality of magnetic microbeads can include various biocompatible magnetic materials, such as, but not limited to iron oxide based magnetic material (e.g., magnetite (Fe₃O₄), maghemite (Fe₂O₃), or combinations of these. The magnetic materials for the microbeads should be non-toxic to cells. The magnetic microbeads of the present disclosure are adapted for conjugation with white blood cells (WBCs) such that the microbeads bind/conjugate with WBCs in the sample and do not substantially bind/associate with the CTCs in the sample (e.g., they do not specifically bind/conjugate/associate with the CTCs or other components present in the sample other than WBC's, and any non-specific association with CTCs, if present, is insignificant/negligible). In embodiments the magnetic microbeads are functionalized for specific binding to WBCs, such as by surface-functionalization with one or more binding agent(s) for specific binding to WBCs but that will not substantially bind the CTCs (e.g., streptavidin/avidin conjugated to biotinylated white blood cell-specific antibodies). In embodiments the magnetic microbeads comprise streptavidin coated magnetic Dynabeads (Life Technologies, Carlsbad, CA). In embodiments, the streptavidin coated magnetic beads are further conjugated to white-blood cell specific antibodies for specific binding to white blood cells. Examples of white blood cell-specific antibodies include, but are not limited to antibodies to CD45, CD45RA, CD66b, CD16, and CD3, and combinations thereof (while other antibodies can be used, a combination of the above antibodies will target substantially all WBC's present in a blood sample). Preferably one or more or two or more WBC biomarker antibodies are used, and in some embodiments all 5 of ate above are used.

In embodiments, the biological sample is whole blood. In embodiments, the sample is whole blood that has been treated with a lysis buffer to lyse/remove red blood cells. In embodiments, the biological sample is combined with the magnetic microbeads to label the WBCs in the sample prior to introduction to the device of the present disclosure to produce a magnetically labeled biological sample, such that the WBCs in the sample are substantially conjugated to magnetic microbeads prior to introduction. In embodiments, about 95% to 99.9%, or more of the white blood cells are conjugated to one or more magnetic microbeads. In embodiments, a majority of white blood cells in the magnetically labeled sample are conjugated to about 2 or more magnetic microbeads. In embodiments, a majority of white blood cells in the magnetically labeled sample are conjugated to about 1 to about 60 magnetic microbeads. In embodiments, the white blood cells in the magnetically labeled sample are conjugated to an average of about 20-50 magnetic microbeads. As used herein, the terms “magnetically labeled biological sample” and “magnetically labeled sample” refer to embodiments described above where a biological sample is combined with magnetic microbeads adapted for specific conjugation with WBC's and not to CTC's or other components of the sample, which are not substantially associated with the magnetic microbeads or otherwise magnetically labeled. Thus, the terms “magnetically labeled biological sample” and “magnetically labeled sample” should not be interpreted to indicate any magnetic labeling of any other components of the biological sample other than the WBCs.

In embodiments, the biocompatible ferrofluid of the present disclosure is a colloidal suspension of magnetic nanoparticles, coated by a biocompatible surfactant and suspended in a carrier fluid. The ferrofluid of the present disclosure is biocompatible and non-toxic to CTCs. In embodiments, the magnetic nanoparticles are a non-toxic magnetic material, such as, but not limited to iron oxide materials (e.g., magnetite (Fe₃O₄), maghemite (Fe₂O₃), and combinations of these. In embodiments, the magnetic nanoparticles are iron oxide particles (e.g., maghemite (Fe₂O₃)). Materials such as iron, cobalt, cobalt ferrite, and FePt are potentially toxic to cells, but could potentially be used if first rendered biocompatible/nontoxic by biocompatible coatings, etc. The magnetic nanoparticles can have a diameter of about 1-20 nm. In embodiments they have an average diameter of about 8-12 nm (e.g., about 11 nm). In embodiments, the magnetic nanoparticles are coated in a biocompatible surfactant to reduce agglomeration and to increase biocompatibility. In embodiments, the biocompatible surfactant can include electric double layer surfactant, polymer surfactant, inorganic surfactant, or a combination thereof. In embodiments, the surfactant is polymethyl methacrylate-polyethylene glycol (PMMA-PEG). In embodiments, the carrier medium can include biocompatible carrier fluids, such as, but not limited to, water, salt solution, or a combination. In embodiments, the carrier medium is a balanced salt solution, such as Hank's balanced salt solution (HBSS). In an embodiment, the biocompatible ferrofluid includes maghemite nanoparticles (Fe₂O₃) coated with polymethyl methacrylate-polyethylene glycol (PMMA-PEG) and 10% (v/v) 10× Hank's balanced salt solution (HBSS). In embodiments, the pH is about 7, and the osmotic pressure is close to that of a biological (e.g., human) cell. In embodiments, the ferrofluid concentration (volume fraction of magnetic particles) is about 0.005-0.05% and intervening ranges. For instance, in embodiments the ferrofluid concentration can be about 0.015-0.03%. (v/v). In embodiments the concentration is about 0.028%. The viscosity of the biocompatible ferrofluid varies based on the concentration of magnetic particles, the surfactant chosen, as well as the carrier fluid. In embodiments, the viscosity of the ferrofluid is about 0.95 mPa·s to 1.8 mPa·s, such as for instance, 1.7 mPa s⁻¹ at room temperature. In embodiments the saturation magnetization was about 1,107 A m⁻¹ and volume fraction of the ferrofluid was about 0.298% v/v.

In embodiments of systems or kits of the present disclosure, the system may include the fully prepared biocompatible ferrofluid as described above, and/or a prepared ferrofluid along with instructions for diluting the fluid with additional carrier fluid to adjust the concentration/volume fraction of magnetic nanoparticles in the fluid. In embodiments, systems or kits of the present disclosure may include a biocompatible ferrofluid composition that includes the plurality of magnetic nanoparticles and instructions for combining the magnetic nanoparticles/biocompatible superparamagnetic sheathing composition with a biocompatible surfactant and biocompatible carrier fluid to make a biocompatible ferrofluid of the present disclosure. In embodiments, systems/kits of the present disclosure can include the plurality of magnetic nanoparticles and the biocompatible surfactant (separately or mixed (e.g., such that the surfactant coats the magnetic nanoparticles) and instructions for combining the biocompatible superparamagnetic sheathing composition with a biocompatible carrier fluid and/or a patient sample to make a biocompatible superparamagnetic sheathing fluid of the present disclosure.

Integrated Inertial Ferrohydrodynamic Cell Separation (i²FCS) Unit

In embodiments, systems and devices of the present disclosure include a cell separation unit that has an integrated inertial focusing stage and a ferrohydrodynamic separation stage, as well as an inlet, filter stage/region, and at least a waste outlet for collection of WBCs and a target outlet for collection of an enriched sample having a greater concentration of rare cells and a lower concentration of WBCs compared to an initial sample.

The device/unit can include at least two stages, although there may be more in some embodiments. Therefore, the terms first, second, third and so-on, when used to describe the stages, should not be considered limiting on the total number of stages but may be used for simplicity to describe the relative ordering of the stages. Additional stages, not explicitly described, may in some aspects appear before the first stage.

The i²FCS units of the present disclosure comprise a sample inlet configured to receive a mixed ferrofluid biological sample comprising a biocompatible ferrofluid combined with a biological sample comprising target rare cells and magnetically labeled WBCs. After the sample inlet, the i²FCS unit includes a filter section fluidly connected to the sample inlet. The filter section includes a first microfluidic channel and one or more filters configured to remove a first plurality of waste particles from the mixed ferrofluid biological sample. The filter section is fluidly connected to the sample inlet and has a first microfluidic channel and one or more filters configured to remove a first plurality of waste particles from the mixed ferrofluid biological sample. In embodiments, the one or more filters are configured to remove a first plurality of waste particles from the biological sample (e.g., large particulate/agglomerated matter in the sample). After the first filter section, the filtered biological sample proceeds to the first separation stage.

After the filter section the units/devices of the present disclosure include an inertial focusing stage. The inertial focusing stage of the units, devices, systems, and methods of the present disclosure differs from the first generation inertial focusing cell separation (iFCS) devices and methods described in PCT/US22/70512, incorporated herein by reference. First, the first generation iFCS device was for separation of completely unlabeled samples based on particle size, which missed some target CTCs closer in size to some WBCs, vs the use of magnetic labeling and separation of WBCs in the present disclosure. The difference in principles of separation are illustrated in FIGS. 1A-1B. Changes in in the dimensions of the device and other applications allowed elimination of the need for sheath flow and a separate sheathing fluid and sheathing fluid inlet, as well as the need for multiple outlets to collect varying size particles. Changes in the geometry of the inertial focusing parts affects the width of the sample stream and starting position of the stream in the ferrohydrodynamic separation portion of the device, which improved separation performance without the need for sheath flow. Also, significant re-design of the outlet collection channel and geometry ensures more complete recovery of all CTCs. The channel geometry in all the parts of the new i²FCS device of the present disclosure also determined new, high throughput flow rates and ferrofluid concentration.

The inertial focusing stage of the i²FCS microfluidic unit of the present disclosure is fluidly connected to the filter section. The first microfluidic channel of the filter section splits into two or more sigmoidal microchannels (as shown in FIG. 3A) with a plurality of alternating micro-curves (such as illustrated in FIG. 3C), each sigmoidal microchannel configured to focus cells within the sample into a narrow stream to produce a focused fluid sample stream. The number of alternating micro-curves in each of the sigmoidal microchannels can be about 30-40 micro-curves. As shown in the embodiment illustrated in FIG. 3C, the alternating micro-curves comprise alternating small and large micro-curves. The diameter under each large micro-curve is about 600-2400 μm and the diameter under each small micro-curve is about 300-800 μm. In embodiments, an interior channel width of each serpentine focusing channel varies along the length of said channel, where the interior channel width at a crest portion of each smaller micro-curve is about 150-400 μm and the interior channel width at a crest portion of each larger micro-curve is about 330-850 μm. In embodiments these dimensions of the micro-curves in sigmoidal microchannels ensure that at a flow rate of about 500-1200 μL/min the position of the focused sample stream as it exits the inertial focusing stage and enters the ferrohydrodynamic separation channel of the ferrohydrodynamic separation stage the starting position of the sample stream is about 200-400 μm away from the side wall of the ferrohydrodynamic separation channel.

After the inertial focusing stage, the i²FCS microfluidic unit of the present disclosure includes a ferrohydrodynamic separation stage fluidly connected to the inertial focusing stage such that the focused fluid sample streams from the two or more sigmoidal microchannels flow into a ferrohydrodynamic separation channel, as illustrated in FIG. 1A. As illustrated in FIG. 3A, various ranges for the geometry and dimensions of the ferrohydrodynamic separation stage are provided. In some embodiments, the internal channel width of the ferrohydrodynamic separation channel is about 800-1600 μm. Also, in embodiments, the ferrohydrodynamic separation channel can have a u-shaped curve, where the length of the channel before the curve is about 51000-59500 μm and the length after the curve is about 51500-60000 μm. Applicant notes that any of the different stages and microfluidic channels of the device may have a larger curve, corner, or bend (distinguishable from the micro-curves in the inertial focusing section). In embodiments, the channel height in the inertial focusing stage and the ferrohydrodynamic separation phase is about 30-300 μm.

The i²FCS microfluidic unit of the present disclosure also includes a waste outlet fluidly connected to and axially aligned with the ferrohydrodynamic separation channel to receive materials flowing through a central portion of the channel. This device/unit also includes one or more target cell outlets each fluidly connected to the ferrohydrodynamic separation channel and offset from the center of the channel and configured to receive material flowing near the outer portion of the channel. An illustration of an embodiment of the geometry of the unit at the end of the ferrohydrodynamic separation channel and the fluid connection to the waste outlet and the one or more target cell outlets is provided in FIG. 3D.

The i²FCS microfluidic unit of the present disclosure also includes one or more magnetic sources adjacent to the ferrohydrodynamic separation stage and configured to produce a substantially symmetric magnetic field having a field maximum along a length of the ferrohydrodynamic separation channel sufficient to cause the white blood cells conjugated to the magnetic beads in the ferrohydrodynamic separation channel to be focused toward a center of the channel and to exit the channel via the waste outlet. The substantially symmetric magnetic field is also configured to cause target rare cells to be deflected towards an outer portion of the ferrohydrodynamic separation channel and to exit the channel via the one or more target cell outlets. In embodiments, the magnetic source comprises an array of magnets comprising a first array and second array, wherein the ferrohydrodynamic separation stage is sandwiched between and substantially centrally aligned between the first magnet array and the second magnet array, wherein the magnets in the first array are oriented to repel the magnets in the second array. An embodiment of the arrangement of the magnets with respect to the ferrohydrodynamic separation stage is illustrated in FIG. 3B. In embodiments, the array of magnets includes six magnets arranged in a sextuple configuration. In some embodiments, a distance between a center of the ferrohydrodynamic separation channel and a magnet junction of the array of magnets is from about 0-50 μm. The array of magnets can be configured to produce a magnetic flux density of about 1.0-3.0 T, such as about 1.1-1.4 T (depending on thickness of the microchannel walls), within the ferrohydrodynamic separation channel and a magnetic flux gradient of about 500-5000 T/m, such as about up 670 T m⁻¹.

In embodiments of the microfluidic device of the present disclosure, the rare cells (e.g., CTCs) that exit via the one or more target cell outlets comprise about 95% or more, 97%, or more, or 99% or more of the total number of rare cells present in the biological sample inserted into the sample inlet when in operation.

### Chemotactic Cell Migration Unit:

Systems and devices of the present disclosure include a chemotactic cell migration unit to further characterize the enriched rare cells after exiting the i²FCS unit described above. In embodiments chemo-modulatory compounds are used to help further characterize the migratory phenotypes of the enriched rare cells to determine various phenotypes such as migratory phenotypes that might be associated with a more invasive cell type posing a greater health risk to a patient, as described in greater detail in the Example below. An embodiment of a design of a chemotactic cell migration unit is illustrated in FIG. 2. Also, U.S. application Ser. No. 16/986,930, incorporated herein by reference, describes various embodiments of a first generation chemo-modulatory devices for enrichment, separation and subtyping of some target cells. The chemotactic cell migration units of the present application have different configurations and geometry and operation parameters than the first generation devices. The chemotactic migration units of the present disclosure are described in greater detail in the Example below.

In embodiments, the chemotactic cell migration units of the present disclosure is designed to be used with one or more chemo-modulatory compounds that affect the migratory behavior of one or more different phenotypes of target cells. For instance, the one or more chemo-modulatory compounds can include at least one compound that is a chemoattractant for CTCs having an invasive phenotype. In other embodiments, the chemoattractant for CTCs is selected from the group consisting of Fetal Bovine Serum (FBS), epidermal growth factor (EGF), and basic fibroblast growth factor (bFGF). In some other embodiments, the one or more chemo-modulatory compounds include at least one compound that is a migration inhibitor for WBCs, such as, but not limited to Slit2.

The chemotactic cell migration unit/device includes various features. It includes an enriched sample inlet to receive an enriched biological sample from the i²FCS unit. It also includes a chemo-modulatory fluid inlet to receive a chemo-modulatory fluid comprising one or more chemo-modulatory compounds. In embodiments, the sample inlet and chemo-modulatory fluid inlet are located on opposite sides of the unit. In embodiments, the chemotactic cell migration unit also includes one or more sample flow channels fluidly connected to the enriched sample inlet and configured to flow the enriched biological sample. The unit/device also includes one or more chemo-modulatory channels fluidly connected to the chemo-modulatory fluid inlet, configured to flow the chemo-modulatory fluid. The flow of the chemo-modulatory fluid from the chemo-modulatory fluid inlet through the chemo-modulatory channels of the device can create a chemogradient within the device or areas of the device. In embodiments, the chemo-modulatory channel(s) are oriented substantially parallel to the one or more sample flow channels. Due to the orientation of the inlets and the flow of the fluids, the enriched sample fluid may, at least initially, flow in an opposite direction of the flow of chemo-modulatory compounds from the chemo-modulatory fluid inlet.

In addition to the sample flow channel and the chemo-modulatory channels, the cell migration unit also includes a plurality of migration microchannels connecting at least one sample flow channel to at least one chemo-modulatory channels. Each of the plurality of migration microchannels oriented substantially perpendicular to the sample flow channel and the chemo-modulatory channel and in fluidic communication with both the sample flow channel and the chemo-modulatory channel. The migration microchannels are small so that fluid flows between the chemo-modulatory channels toward the sample flow channel to create a chemo-gradient, and only about 1 cell can pass through the migration microchannel at a time. In embodiments, the height (or width) of each migration microchannel is smaller than the height (or width) of each of the sample flow channel and the chemo-modulatory channels. In embodiments, each migration microchannel has dimensions to allow migration of about a single cell at a time, for instance in embodiments, each migration microchannel has a width of about 7-50 μm (e.g., about 30 μm), a height of about 3-8 μm (e.g., about 5 μm), and a length of about 200-2000 μm.

The cell migration unit also includes at least one migratory target cell outlet at and end of a chemo-modulatory channel opposite the chemo-modulatory fluid inlet configured to collect migratory target cells that have migrated from the enriched sample inlet. As described above, the distance and speed with which a target rare cell has migrated with respect to the chemo-modulatory fluid within the device can differentiate the cells into different migratory phenotypes. For CTCs, more invasive phenotypes have also been shown to have more invasive characteristics and thus this can provide additional diagnostic approaches as well as novel ways to investigate phenomena such as epithelial to mesenchymal transition that appears to accompany a transition to a more high-risk invasive phenotype.

### Combined Devices and Systems of the Present Disclosure

Embodiments of the present disclosure include combined integrated inertial ferrohydrodynamic cell separation (i²FCS) and cell migration devices that incorporate the i²FCS unit of the present disclosure as well as the chemotactic cell migration units of the present disclosure. The i²FCS unit can be used according to methods of the present disclosure to produce an enriched biological sample that has an enriched population of rare cells, such as CTCs. Then the combined device also includes an integrated chemotactic cell migration unit for further characterizing the target cells in the enriched sample as described above. While the units can function independently, the combined device includes the two units in an integrated or conjoined device. In embodiments, the two units can be included on a same microfluidic platform or on separate substrates where the enriched sample is transferred from one unit to the next

Cell separation systems of the present disclosure include the i²FCS unit as well as the chemotactic cell migration unit of the present disclosure along with other components useful for operation of the system. One other such component is a plurality the magnetic microbeads form producing the magnetically labeled biological sample (with magnetically labeled WBCs) as well as the biocompatible ferrofluid (or ingredients to make it). Another feature of some systems of the present disclosure is one or more chemo-modulatory compounds for use in the cell migration unit. The chemo-modulatory compounds are as described above, compounds that affect the migratory behavior of one or different phenotypes of target cells.

### Additional Descriptions

The present disclosure also includes methods described above using the microfluidic devices of the present disclosure and the systems of the present disclosure (e.g., microfluidic device plus biocompatible superparamagnetic sheathing fluid and magnetic microbeads) to enrich CTCs in a biological sample. In some aspects, the methods are capable of isolating a majority of the unlabeled rare cells. In some aspects, the unlabeled rare cells are circulating tumor cells in a whole blood sample, and the majority of the circulating tumor cells comprises about 90%, about 95%, about 97%, about 99%, or more of the circulating tumor cells as compared to a total number of circulating tumor cells present in the biological sample inserted into the first fluid inlet when in operation.

In some aspects, the biological sample includes whole blood, wherein the whole blood includes a plurality of components. In some aspects, the plurality of components comprises magnetically labelled white blood cells, and wherein at least 95%, at least 98%, at least 99%, at least 99.9%, or more of the white blood cells are not collected in the one or more circulating tumor cell outlets as compared to a total number of white blood cells present in the whole blood inserted into the first fluid inlet when in operation. This can mean, for instance, that at least 95%, at least 98%, at least 99%, at least 99.9%, or more of the white blood cells are collected in one or more of the filters and the waste outlet as compared to a total number of white blood cells present in the whole blood inserted into the sample fluid inlet when in operation. This can result in, for example, that at least 90%, 92%, 95%, or more of the unlabeled rare cells are collected in the one or more circulating tumor cell outlets as compared to a total number of unlabeled rare cells present in the whole blood inserted into the sample inlet when in operation.

Methods are provided for enriching, separating, or isolating unlabeled rare cells such as circulating tumor cells from a sample, e.g., a biological sample such as whole blood. In some aspects, the biological sample is or includes whole blood. In some embodiments the biological sample includes whole blood treated with a lysis buffer to lyse red blood cells as described above. In some aspects, the biological sample includes about 1 to 1000 circulating tumor cells per milliliter of the biological sample, including both natural samples and samples spiked with CTCs for research purposes. In embodiments, the biological sample comprises about 1-10 circulating tumor cells per milliliter of the biological sample. Examples of the circulating tumor cells can include those selected from the group consisting of a primary cancer cell, a lung cancer cell, a prostate cancer cell, a breast cancer cell, a pancreatic cancer cell, and a combination thereof.

In embodiments, the biological sample is a sample from an animal host (e.g., a mammal, human, etc.). In embodiments, the biological sample is a fluid having a plurality of components (e.g., cells, fluids, etc.) (e.g., blood, urine, saliva, exudate, homogenized tissue in a biocompatible fluid, etc.). In embodiments, the biological sample is from a human. In embodiments, the biological sample is whole blood or whole blood that has been treated with lysis buffer to lyse red blood cells. In embodiments, the biological sample is whole blood, or RBC lysed blood from a human host having cancer or suspected of having cancer.

Untreated biological samples have various amounts of circulating tumor cells, dependent on the type of cancer, the stage of cancer, other cancer treatments, etc. In general, un-spiked samples from a host have about 1-10 circulating tumor cells/ml of the biological sample. However, for research purposes, sometimes samples are spiked with additional CTCs. Also the level of CTCs can be higher depending on the tumor grade of a host. Thus, in embodiments, the biological sample can have from about 1 to about 1000 circulating tumor cells/ml of biological sample. It will be understood that for research purposes even higher amounts of circulating tumor cells may be present in a sample and still be within the scope of the present disclosure. However, unlike some previous methods, the present methods/devices/systems are able to detect/enrich CTCs from a sample having 10 or fewer CTCs/ml of biological sample.

In embodiments, the CTCs can include, but are not limited to, a primary cancer cell, a lung cancer cell, a prostate cancer cell, a breast cancer cell, a pancreatic cancer cell, and a combination thereof.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure.

### Example 1 Simultaneous Biochemical and Functional Phenotyping of Single Circulating Tumor Cells Using Ultrahigh Throughput and Recovery Microfluidic Devices

Profiling circulating tumor cells (CTCs) in cancer patients' blood samples will lead to greater understanding of the complex and dynamic nature of metastasis. This task is challenged by the fact that CTCs are not only extremely rare in circulation but also highly heterogenous in their molecular programs and cellular functions. The present example describes an embodiment of a combinational approach for the simultaneous biochemical and functional phenotyping of patient-derived CTCs, using an integrated inertial ferrohydrodynamic cell separation (i²FCS) method and a single-cell microfluidic migration assay. This combinatorial approach offers unique capability to profile CTCs on the basis of their surface expression and migratory characteristics. We achieve this using the i²FCS method that successfully processes whole blood samples in a tumor-cell-marker and size-agnostic manner. This i²FCS method enables an ultrahigh blood sample processing throughput of up to 2×10⁵ cells s⁻¹ with a blood sample flow rate of 60 mL h⁻¹. Its short processing time (10 minutes for a 10 mL sample), together with a close-to-complete CTC recovery (99.70% recovery rate) and a low WBC contamination (4.07-log depletion of leukocytes), result in adequate and functional CTC for subsequent studies in the single-cell migration device. For the first time, this example describes this novel approach to query CTCs with single-cell resolution in accordance with their expression of phenotypic surface markers and migration properties, revealing the dynamic phenotypes and the existence of a high-motility subpopulation of CTCs in blood samples from metastatic lung cancer patients. This method can be applied to study the biological and clinical values of invasive CTC phenotypes.

### Introduction

Circulating tumor cells (CTCs) are implicated in the formation of metastatic tumors, which is responsible for as much as 90% of cancer-related mortality.1-6 While the number of tumor cells in blood circulation has been correlated to clinical outcomes,7-9 it has become clear that enumeration of CTCs alone is not sufficient in understanding their multifaceted role in metastasis, in which CTCs participate in nearly all aspects of the process.3 ⋅ 10⋅ 11 Cancer patients have CTCs of varying phenotypes in their blood circulation; 1⋅ 4 ⋅ 10⋅ 12-15 while some cells passively detach themselves from the primary tumor,16 a fraction of them gain the ability to actively invade distance organs through modifying their cellular programs, morphology and surrounding tissues.17 Cells of this invasive phenotype often exhibit a high-motility trait that facilitates hematological spread, giving them the greatest threat of metastasis.3⋅ 10⋅ 11 ⋅ 19 Despite rapid advances in the understanding of the molecular mechanisms of CTCs,4⋅ 13⋅ 15 functional properties of invasive CTC phenotype(s) remain poorly understood due to the limitations of existing CTC isolation and phenotyping methods.20-22

CTC's extreme scarcity in blood circulation (<10 CTCs per one milliliter of whole blood) and a lack of methods for the isolation of adequate and functional cells represent a significant bottleneck in studying the invasive phenotypes of CTCs.20⋅ 22 CTCs are highly heterogeneous in their biological and biophysical characteristics with multiple phenotypes co-existing, which can evolve dynamically over the course of metastasis.3⋅ 10⋅ 11 Existing isolation techniques relying on the expression of tumor cell surface epitopes bias the sampling population and reduce the heterogeneity of captured cells.20 These techniques also lead to immobilized and non-functional CTCs and limit possibility of conducting functional studies.20 Physical property separation methods relying on size-based selection can separate larger CTCs from smaller leukocytes without limiting to molecular markers for selection. However, the isolated cells are contaminated with a large number of leukocytes and may also miss CTCs that were morphologically similar to the leukocytes. As such, current microfluidic methods for invasiveness phenotyping of tumor cells were mostly confined to cultured cancer cells rather than patient-derived CTCs.23-27 New methods are needed to isolate adequate and functional CTCs from patient samples so that the properties of invasive cells can be identified and characterized.

Here we report a novel combinational approach, which first uses an integrated inertial ferrohydrodynamic cell separation (i²FCS) method to recover virtually all CTCs from blood samples with minimal contamination using an approach that is not reliant on tumor cell markers and cell size for separation. Adequate and functional CTCs isolated from this method enable quantitative profiling of their biochemical and functional properties using a microfluidic assay that can track single tumor cell's chemotactic migration over time. In isolating CTCs when they are present at extremely low levels in the whole blood, we find that i²FCS method enables an ultrahigh blood sample processing throughput of up to 2×10⁵ cells s⁻¹ with a sample flow rate of about 60 mL h⁻¹, resulting in an approximate 10 minute device processing time for a standard 10 mL of blood sample. The short processing time, together with a close-to-complete CTC recovery rate of 99.70% and a low WBC contamination of ˜507 WBCs carryover per milliliter blood processed, preserve isolated CTCs' viability and biological functions, allowing simultaneous biochemical and functional phenotyping of single tumor cells isolated from cancer patient's blood. Using this approach, we reveal a great diversity of biochemical and functional phenotypes of CTCs with single-cell resolution. CTCs with different levels of epithelial and mesenchymal marker expression exhibit varying chemotactic migration profiles, and there exists a high-motility subpopulation of CTCs in the patient's sample.

### Results and Discussion

Overview of the i²FCS Approach

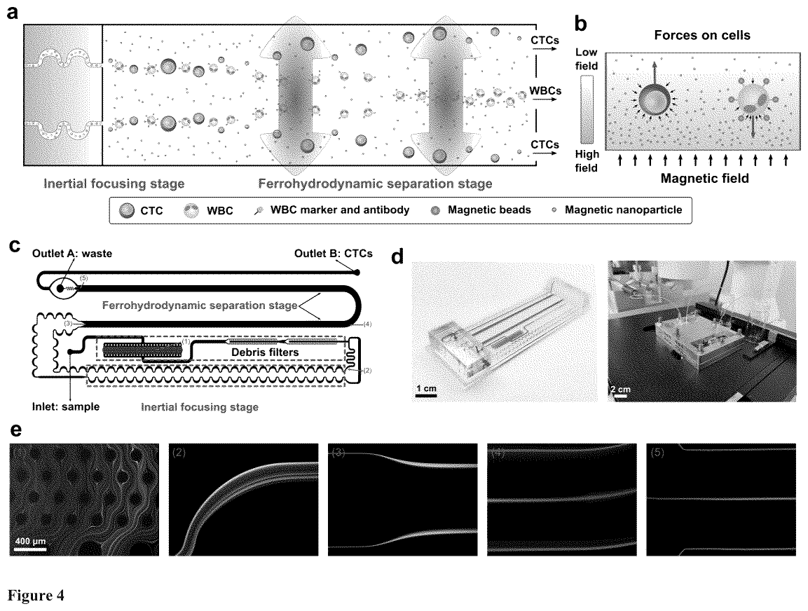

The integrated inertial ferrohydrodynamic cell separation (i²FCS) approach leverages the integration of cell size-based inertial focusing and cell magnetization-based ferrohydrodynamic separation (FIG. 4a) for a tumor-cell-marker-and-size-agnostic isolation. In this approach, a red blood cell-lysed blood sample from cancer patients is treated to label the white blood cells (WBCs) and is mixed with a colloidally stable magnetic fluid (ferrofluid) first flow through an inertial focusing stage, in which both tumor cells and blood cells are ordered into narrow streams in sigmoidal microchannels with alternating curvatures. The channel geometry and flow parameters in this stage enable the cells to experience inertial lift and Dean drag that force them to migrate to balanced locations within the curved channel (FIGS. 4a and 4e).28-30

In the second stage of the approach, inertially-focused cell streams are ferrohydrodynamically separated into different spatial locations according to their magnetization difference. Its physical principle, illustrated in FIG. 4b, shows that white blood cells (WBCs) are rendered magnetic by labeling of magnetic microbeads through a combination of leukocyte biomarkers, while CTCs remain unlabeled. Magnetization of the ferrofluid is fine-tuned to be less than that of WBC-bead conjugates, so that unlabeled CTCs with a close to zero magnetization, regardless of their size profiles, are collected via a magnetic field minima close to the boundary regions of the microchannel due to a phenomenon known as “diamagnetophoresis”,31 while WBC-bead conjugates are depleted via a magnetic field maxima at the channel center through a competition between both “magnetophoresis” and “diamagnetophoresis” (FIG. 4e). The integration of inertial focusing and ferrohydrodynamic separation results in a compact microfluidic device with just one fluidic inlet and two fluidic outlets (FIG. 4c), which can be operated using a single syringe pump for CTC isolation (FIG. 4d).

Design Principles of the i²FCS Approach

In the present example, the i²FCS approach was optimized to realize an isolation of functional CTCs in a tumor cell marker and size agnostic manner. Optimized i²FCS devices have the following characteristics: (1) a nearly complete isolation of CTCs from blood samples with 99.70% recovery rate; (2) an ultrahigh throughput of >600 millions of nucleated cells per hour (up to 200,000 cells s⁻¹) and a ultrahigh sample flow rate of 60 mL h⁻¹; (3) an extremely low carryover of ˜507 WBCs for every 1 mL of blood processed; (4) isolated CTCs preserving their initial viability and functions and enabling their biochemical and functional analysis. These performance characteristics were realized through optimizing i²FCS devices' geometry, magnetic field pattern, WBC functionalization, sample flow rate and ferrofluid concentration. A physical model that could predict the dynamics of cells in the i²FCS devices was developed for the optimization process.32⋅ 33

Firstly, the channel dimensions of both inertial focusing and ferrohydrodynamic separation stages in i²FCS were designed to accommodate a high blood sample flow of 60 mL h⁻¹, which greatly reduced the device processing time of blood samples (10 minutes for a standard 10 mL blood sample). For the inertial focusing stage, we designed it so that both tumor and blood cells with diameters larger than 4 μm could be efficiently focused at a flow rate of 60 mL h⁻¹. The geometry of the inertial focusing stage was fine-tuned so that the particle Reynolds number (Rₚ) was about 5.4, and the channel Reynolds number (R_{c}) was about 51.5 when the flow rate was about 60 mL h⁻¹, ensuring a well-focused cell stream (˜100 μm in width) before the ferrohydrodynamic separation stage. For the ferrohydrodynamic separation stage, the channel dimension (about 54.8×about 1.2×about 0.06 mm, length×width×height) was optimized so that the channel Reynold's number was about 21.3 when the sample flow rate was about 60 mL h⁻¹, ensuring unperturbed laminar flow conditions during CTC isolation.

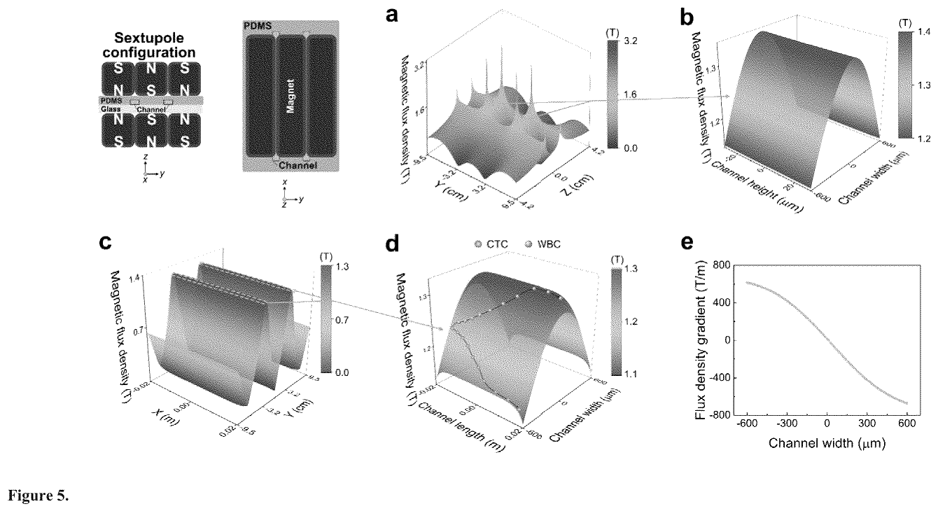

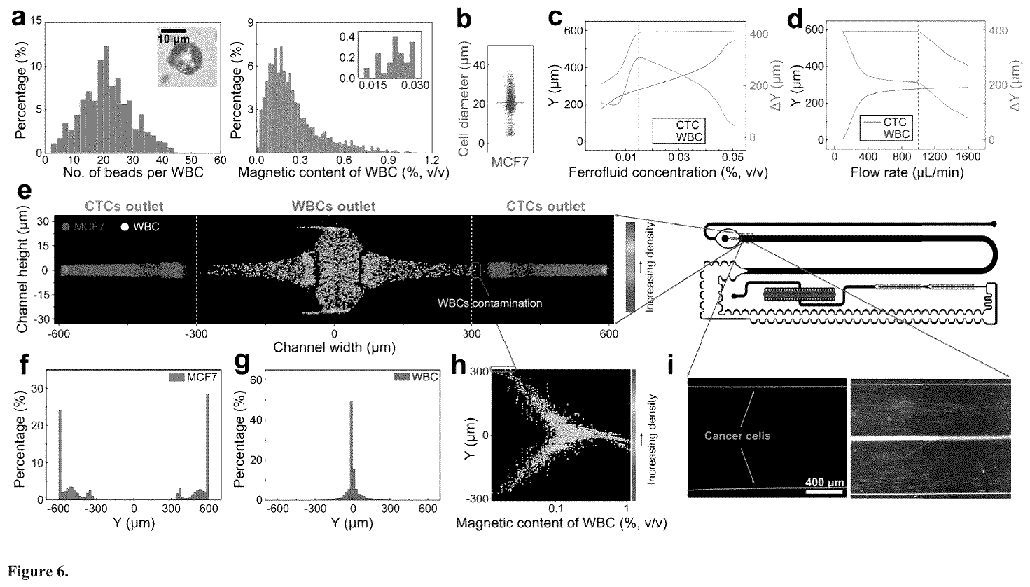

Secondly, we designed the generation of magnetic fields in i²FCS with a sextuple magnet configuration (FIG. 5) to obtain a significant magnetic force on the cells for efficient cell separation. A magnetic flux density of up to about 3.2 T (1.1-1.4 T within the ferrohydrodynamic separation channel) (FIGS. 5a-d) and a gradient of magnetic flux of up to about 670 T m⁻¹ (FIG. 5e) were obtained from the sextuple configuration. As shown in FIGS. 5b and 5d, the magnetic flux density was maximal at the center of the separation microchannel, while the absolute value of the flux density gradient was minimal. Using this magnetic field pattern, the directions of the magnetophoretic WBCs and diamagnetophoretic CTCs in the microchannel are opposite to each other, eliminating the need of sheath flow in i²FCS devices and simplifying the device's fluidic operation. Thirdly, we optimized the WBC functionalization by using a combination of five leukocyte biomarkers (CD45, CD45RA, CD66b, CD16, and CD3).34 Biotinylated biomarker antibodies were labeled with the WBCs then conjugated with streptavidin-coated Dynabeads (1.05 μm diameter, 11.4% volume fraction of magnetic materials). The use of five markers allowed us to reduce the number of Dynabeads per WBC (20 per cell), because on average streptavidin-coated Dynabeads had a high probability of conjugating to WBCs due to the increased presence of biotins from the five markers. In our experiences, unconjugated Dynabeads tended to clog microchannels under strong magnetic field gradients. Therefore, the decreased use of Dynabeads in this method resulted in the elimination of microchannel clogging issues. With this labeling protocol, WBCs were conjugated with an average of about 21±9 (mean±s.d.) beads and >99.95% of WBCs were labeled with at least two beads (FIG. 6a, left). Based on the number of beads on the WBCs and corresponding cell size, we calculated the upper bound of the magnetic volume fraction of the ferrofluid to deplete WBCs. Lastly, we studied the effects of ferrofluid concentration and blood sample flow rate on the separation performance in the above-mentioned physical model. Simulated cells' position (denoted as Y) and separation distance between WBCs and tumor cells at the device outlets (denoted as ΔY) on the ferrofluid concentration and sample flow rate are shown in FIGS. 6c and 6d. Maximal separation distance occurred when the ferrofluid concentration is about 0.015% (FIG. 6c) and the flow rate was about 1000 μL min⁻¹ or about 60 mL h⁻¹ (FIG. 6d). Using these optimized parameters (ferrofluid concentration: 0.015% (v/v); flow rate: 1000 μL min⁻¹ or 60 mL h⁻¹), positions of 10,000 MCF7 cancer cells and 10,000 labeled WBCs at the outlet of the device were simulated and shown in FIG. 6e. 100% of the MCF7 breast cancer cells are deflected toward the channel walls and are collected from the CTCs outlet of the device (FIG. 6f), while approximately 99.95% of WBCs are depleted through the WBCs outlet (FIG. 6g).

Throughput, Recovery, Purity and Biocompatibility of the i²FCS Approach

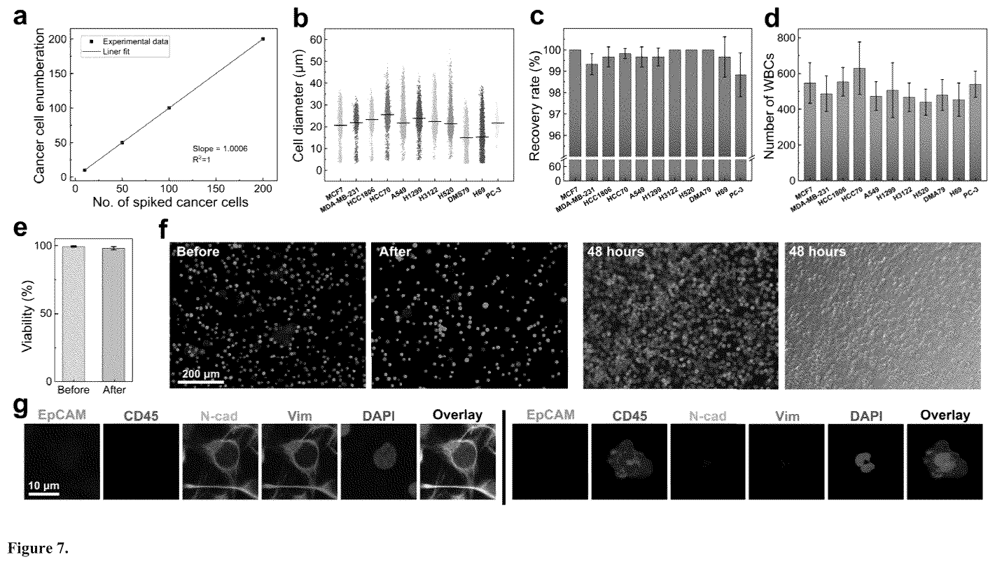

Using the optimized i²FCS device and operating parameters, we validated it with spiked cancer cells from a total of 11 cultured cancer cell lines, including 4 breast cancer cell lines (MCF7, MDA-MB-231, HCC1806, HCC70), 4 non-small cell lung cancer cell lines (A549, H1299, H3122, H520), 2 small cell lung cancer cell lines (DMS79, H69), and 1 prostate cancer cell line (PC-3). We evaluated the performance of i²FCS in the cancer cell isolation, including sample flow rate and cell-processing throughput, cell recovery rate, WBC contamination, viability and proliferation of isolated cells. FIG. 6i shows a typical separation process, in which approximately 100 MCF7 breast cancer cells stained with green fluorescence were spiked into 1 mL of WBCs (˜6 million cells/mL) and processed at a flow rate of 60 mL h⁻¹. Cancer cells and WBCs were distinctively separated into different streams at the outlets of the device. No channel clogging due to magnetic beads was observed during the device operation of processing up to 600 millions of nucleated cells with a throughput of 100,000 cells s⁻¹ and a flow rate of 60 mL h⁻¹. The throughput and flow rate of i²FCS are approximately one order of magnitude higher than most existing CTC isolation methods (see supplementary information). The ultrahigh throughput of i²FCS enables processing a typical blood sample of 10 mL in 10 minutes, significantly reducing chances of cell apoptosis during the device operation. We further characterized the performance of i²FCS in recovering spiked cancer cells at clinical concentrations (10-200 cells mL⁻¹). MCF7 breast cancer cells with spike ratios ranging from 10 to 200 cells per mL were recovered using the devices at a recovery rate of 100% with minimal variations (n=3, FIG. 7a), indicating i²FCS's ability to completely recover spiked cancer cells at clinical concentrations.

We further challenged the device with 10 additional cancer cell lines with distinct size profiles (FIG. 7b). i²FCS showed close-to-complete recovery rates across all cancer cell lines used in this study (100.00±0.00%, 99.33±0.49%, 99.67±0.47%, 99.83±0.24%, 99.67±0.47%, 99.67±0.42%, 100±0.00%, 100±0.00%, 100±0.00%, 99.67±0.94%, and 98.83±1.03% for MCF7, MDA-MB-231, HCC1806, HCC70, A549, H1299, H3122, H520, DMS79, H69, and PC-3 cell lines, mean±s.d., n=3 for each cell line) (FIG. 7c). The average recovery rate across 11 cancer cell lines was 99.70±0.34% (mean±s.d., n=11), including the small cell lung cancer cells (DMS79 and H69). The recovery rate of i²FCS device is higher than other microfluidic approaches (see supplementary information), including the CTC-iChip.35⋅ 36. Current range of cell concentration processed by i²FCS was 3-20 millions cells/mL. Higher cellular concentration would slightly decrease the cancer cell recovery rate (see supplementary information). i²FCS also greatly reduced the contamination of WBCs. The i²FCS device achieved 4.07-log depletion of WBCs by removing 99.992% of the leukocytes from the blood samples, with approximately 507±53 (mean±s.d., n=3) cells carryover in the CTC collection outlet after processing 1 mL of blood (FIG. 7d). The majority of WBC contamination were WBCs labeled with 1 magnetic bead. The level of WBC contamination found in i²FCS device is significantly lower than the majority of other microfluidic approaches (see supplementary information), and is comparable to the CTC-iChip approach.35⋅ 36

Lastly, we investigated the effect of the device processing on the cells' viability and proliferation. The combination of low ferrofluid concentration (0.015% of magnetic content by volume) and laminar flow conditions in the i²FCS device showed little impact on the viability, intactness and proliferation of the isolated cancer cells. FIG. 7e shows that cell viabilities of H1299 lung cancer cells before and after i²FCS processing were 99.31±0.42% and 98.10±1.35% (mean±s.d., n=3), respectively, indicating a negligible device effect on the cell viability. Fluorescence images of live/dead assay in FIG. 7f show the viability and intactness of the cancer cells were well preserved after the device processing. The isolated cancer cells continued to proliferate into confluence after 48 hours' culture (FIG. 7f), with unaffected marker expressions on their surface (FIG. 7g).

### Biochemical Phenotyping of CTCs in Cancer Patients

To evaluate the performance of i²FCS in isolating heterogeneous CTCs in clinical samples, we conducted a study of samples collected from 2 patients exhibiting stage IV metastatic non-small cell lung cancer. Immunofluorescent staining was used to distinguish CTCs and WBCs, and CTCs of different phenotypes. We used the i²FCS devices to process blood samples from the patients, who were recruited and consented at the University Cancer and Blood Center (Athens, Georgia) under an approved IRB protocol (University of Georgia, VERSION00000869). Surface markers corresponding to epithelial and mesenchymal phenotypes were chosen because CTCs are reported to go through EMT, epithelial to mesenchymal transition, in which original epithelial tumor cells transition into stem-like mesenchymal cells.10 ⋅ 11 ⋅ 37 The loss of epithelial characteristics and the acquisition of mesenchymal characteristics are closely linked to the tumor cells' high motility and invasiveness to create a new tumor site.10 ⋅ 37-39 CTCs of this functional phenotype are therefore the focus of this study. 20 mL of blood sample from each patient was processed by the i²FCS devices. A quarter of the isolated cells were used for biochemical phenotyping through immunofluorescent staining with an epithelial marker (EpCAM) that is downregulated in EMT,20⋅ 37 ⋅ 38 two mesenchymal markers (vimentin and N-cadherin) that are upregulated in EMT, 18⋅ 37⋅ 38 a leukocyte marker (CD45), and a nucleus marker (DAPI) for their identification. WBCs were identified as CD45 positive and DAPI positive (EpCAM−/Vim−/N-cad−/CD45+/DAPI+). CD45 negative and DAPI positive CTCs were classified into three different phenotypes including epithelial phenotype (EpCAM+/Vim−/N-cad−), mesenchymal phenotype (EpCAM−/Vim+/N-cad−, EpCAM−/Vim−/N-cad+, or EpCAM−/Vim+/N-cad+), and mixed epithelial and mesenchymal phenotype (EpCAM+/Vim+/N-cad− or EpCAM+/Vim+/N-cad+).

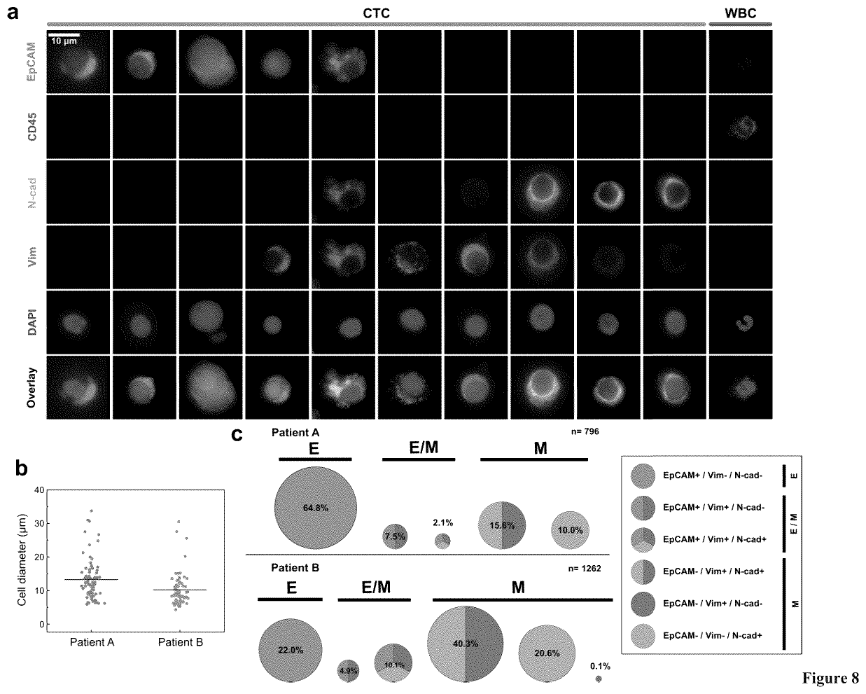

Examples of isolated CTCs are shown in FIG. 8a. We first note that a significant number of CTCs were isolated from both patients' blood samples. 796 cells were identified as CTCs from patient A in a 5 mL volume of blood sample at a concentration of 159 CTCs/mL of blood sample, and 1262 were identified in patient B′ sample (5 mL blood, 252 CTCs/mL concentration). The high counts of CTCs could be explained by the disease stages (stage IV metastatic non-small cell lung cancers) of both patients and the ability of i²FCS to completely recover CTCs from blood. For verification purpose, a blood sample from a third patient (patient C, stage IV lung cancer) was processed by both i²FCS and a recently reported size-selection method (inertial-FCS),40 both of which yielded similarly high counts of CTCs (see supplementary information).

Isolated CTCs from both patients were intact, indicating a minimal impact of the device processing on the cells' morphology. Consistent with previous reports,32⋅ 41-46 the effective cell diameter of isolated CTCs, defined as the maximum Feret diameter of the cells under bright-field imaging, showed a high level of variation for both patients. The effective diameters of randomly selected (n=75) CTCs from patient A's sample were 13.29±6.13 μm (mean±s.d.), with the smallest diameter being 5.88 μm and the largest being 33.74 μm (FIG. 8b). For patient B, the effective diameters of randomly selected CTCs (n=70) were 10.22±4.85 μm (mean±s.d.), where the smallest diameter was 4.28 μm and the largest was 30.51 μm (FIG. 8b). While the clinical relevance of CTCs with varying sizes is unclear, some consider that cells switching from an active state to a dormant state may be the cause of their size variation, which could contribute to their metastatic potential.43 Nonetheless, the polydispersity of isolated cells highlights the effectiveness of the cell size agnostic i²FCS approach in recovering CTCs that are comparable in size to WBCs, enabling downstream studies on these cells.

We further characterized the biochemical phenotypes of the isolated CTCs through their surface antigen expression using the above-mentioned epithelial and mesenchymal markers. The proportion of each phenotypic subtypes of CTCs are summarized in FIG. 8c, which shows interesting comparison between two patients. Isolated CTCs of patient A had a significant portion of epithelial phenotype (64.8% EpCAM+/Vim−/N-cad−) while patient B's CTCs had a predominately mesenchymal phenotype (40.3% EpCAM−/Vim+/N-cad+, 20.6% EpCAM−/Vim−/N-cad+, and 0.1% EpCAM−/Vim+/N-cad−), indicating that the majority of Patient B's cells have gone through the EMT. Patient A also presented 25.6% mesenchymal CTCs (15.6% of EpCAM−/Vim+/N-cad+, and 10.0% of EpCAM−/Vim−/N-cad+) in addition to the epithelial phenotype. Patient B presented 22.0% of epithelial CTCs (EpCAM−/Vim+/N-cad) in addition to the mesenchymal phenotype. Both patients had a relatively small percentage of CTCs that presented mixed epithelial and mesenchymal phenotypes (Patient A: 7.5% EpCAM+/Vim+/N-cad− and 2.1% EpCAM+/Vim+/N-cad+; Patient B: 4.9% EpCAM+/Vim+/N-cad− and 10.1% EpCAM+/Vim+/N-cad+, FIG. 8c). The cells with mixed epithelial and mesenchymal phenotypes likely represented CTCs that were in transition between epithelial and mesenchymal status, indicating their evolution to more invasive phenotypes. Overall, the heterogeneity of biomarker expressions of isolated CTCs from these patients is consistent with previous reports and highlights the maker agnostic isolation of i²FCS approach. CTCs of mesenchymal phenotype are reported to possess high motility and are more invasiveness than the epithelial phenotype.10 ⋅ 37-39 Therefore identifying the invasive subtype of CTCs with high motility is the focus of the subsequent functional study.

### Functional Phenotyping of CTCs in Cancer Patients

Adequate and functional CTCs isolated from the i²FCS approach enable their simultaneous biochemical and functional phenotyping. In this study, we accessed how CTC subpopulations with different levels of epithelial and mesenchymal marker expression affect their chemotactic migration. We chose cell migration to access CTCs' functions because high motility of these cells are implicated in the metastatic spread, including local invasion into surrounding stroma and intravasation into blood circulation, extravasation into parenchyma of foreign tissue, colonization and formation of metastatic lesions.3 ⋅ 10 ⋅ 11 ⋅ 18 ⋅ 19 The identification of high-motility CTCs would facilitate the prediction of a patient's risk of developing metastasis and the design of personalized therapeutics. i²FCS's ultrahigh recovery rate allows us to isolate virtually all CTCs from the patient samples, which potentially contain a subpopulation of these highly motile CTCs. In order to identify this subpopulation, we developed a new microfluidic assay that tracked cells' chemotactic migration with single cell resolution over a 24-hour period in confined microchannels.

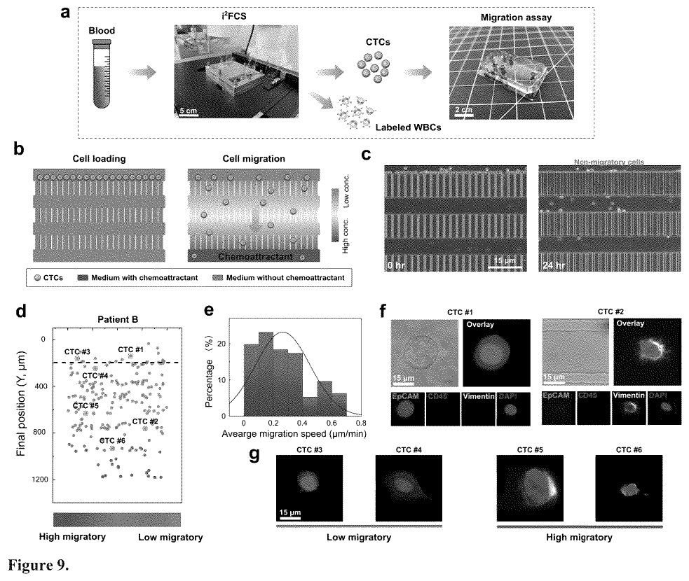

CTC isolation and migration characterization process is shown in FIG. 9a. A 20 mL of blood sample from Patient B was first processed by the i²FCS device to isolate CTCs. Patient A's sample experienced a delay in its processing and was not included in the migration study. The isolated cells from Patient B were divided into three portions, with one quarter of the cells used for biochemical phenotyping through immunofluorescent staining (described above), and one quarter for the microfluidic migration assay. The remaining one half of cells was preserved for future studies.

In constructing the microfluidic device and assay for CTCs chemotactic migration phenotyping, we applied the following design principles. Firstly, we chose to use chemotactic migration to guide CTC's migratory direction in the microfluidic assay because CTCs are most efficient when the cell is involved in directed migration.47 ⋅48 We used a spatial gradient of growth factors including epidermal growth factor (EGF), basic fibroblast growth factor (bFGF) and fetal bovine serum (FBS), to guide CTCs' migration in the microchannels,49 and a spatial gradient of Slit2 to inhibit the migration of carryover WBCs.50 ⋅ 51 The gradient of growth factors were maintained via a continuous perfusion for a 24-hour period in the microchannel to enable chemotactic migration of CTCs (FIG. 9b). Secondly, we constructed microchannels to recapitulate the confined space through which tumor cells infiltrate organs in vivo.19⋅ 52-54 A total of 5,000 single cell migration tracks were packed in the device for CTCs to migrate, with each track having a cross-section of 30 μm (width) by 5 μm (height) and a total length of 1200 μm (FIG. 9b), close to the dimensions of the tunnel-like tracks CTCs encountered in the extracellular matrix (ECM) of the tumor stroma.19⋅ 52-54 The single cell tracks were periodically interrupted to enable collection of migrated cells at the end of the experiments.

This assay was first validated using H1299 lung cancer cells to show that it could differentiate migratory versus non-migratory subtypes (FIG. 9c). In experiments using patient-derived CTCs, cells isolated from Patient B using an i²FCS device were seeded in the microfluidic migration device at the loading channel, and allowed to migrate along the growth factors' gradient for about 24 hours with incubation conditions of about 37° C. and about 5% CO₂. At the end of the 24-hour period, migratory cells were immunofluorescently stained within the device with an epithelial marker (EpCAM), a mesenchymal marker (Vimentin, Vim), a leukocyte marker (CD45) and a nucleus marker (DAPI) to identify the cell types. Migratory distance and speed of each identified CTCs (EpCAM+/Vim−/CD45−/DAPI+, EpCAM−/Vim+/CD45−/DAPI+, or EpCAM+/Vim+/CD45−/DAPI+) in the single cell tracks were recorded and analyzed.

We estimated that a total of approximately 1260 CTCs isolated from a 10 mL blood sample were seeded in the microfluidic migration device at the start of the migration assay. The number of CTCs was calculated from the 252 CTCs/mL concentration obtained through immunochemistry for Patient B′ sample. At the end of the 24-hour migration assay, we identified again through immunochemistry that a small percentage of initial CTCs (16.4%, 207 out of the 1260 seeded cells) remained in the migration device and exhibited chemotactic migration towards the growth factors' gradient. The other 83.6% of CTCs were likely apoptotic and washed away by the perfusion within the assay timeframe. FIGS. 9d-f summarize the distributions of final migratory position, migration speed and surface marker expression of the high-motility cell subpopulation. The migration speed of individual cells was calculated from the distance migrated (difference between initial and final positions within the microchannel) within 24 hours. These high-motility cells exhibited variable levels of migration during the 24-hour period, with a mean speed (FIG. 9e) of 0.26±0.19 μm min⁻¹ (mean±s.d., n=207). This speed indicated that the migratory CTCs likely utilized the mesenchymal locomotion in the microchannels, which was reported to have a speed range of 0.1-1 μm min⁻¹.55 We also observed that cells with a faster migratory speed and a longer migratory distance tended to have elongated cell morphology, while cells with a slower migratory speed and a shorter migratory distance had a mostly rounded shape (FIGS. 9f-g), consistent with previous findings of mesenchymal migration.56 ⋅ 57 Through the single-cell migration assay, we identified a subpopulation of CTCs from Patient B's sample that possessed high motility towards the gradient of growth factors. This subtype of high-motility CTCs exhibits different levels of epithelial and mesenchymal marker expressions and varying chemotactic migration property. The identification of these high-motility CTCs enables further molecular and functional studies on them.

Comparison of i²FCS to Existing CTC Enrichment Methods

We objectively evaluated i²FCS's performance in CTC separation to existing methods, using four commonly used metrics in calibrating CTC isolation methods, including the cell-processing throughput, CTC recovery rate, WBC contamination or carryover at device output and integrity of enriched cells. i²FCS method reported an ultrahigh blood sample processing throughput of up to 2×10⁵ cells s⁻¹ with a blood sample flow rate of 60 mL h⁻¹. It resulted in a close-to-complete recovery of spiked cancer cells (99.70% recovery rate) and an ultralow WBC contamination (4.07-log depletion of leukocytes, removing 99.992% of the leukocytes from the blood samples, with approximately 507 WBC carryover per 1 mL of processed blood). The short processing time of i²FCS (10 minutes for 10 mL of blood) and complete recovery of CTCs produced adequate, viable and functional cells for subsequent cell-migration studies. We compared iFCS's performance to a total of 49 recently published CTC separation methods (see supplementary information) and found that i²FCS had better overall performance in the above-mentioned four metrics than existing methods.

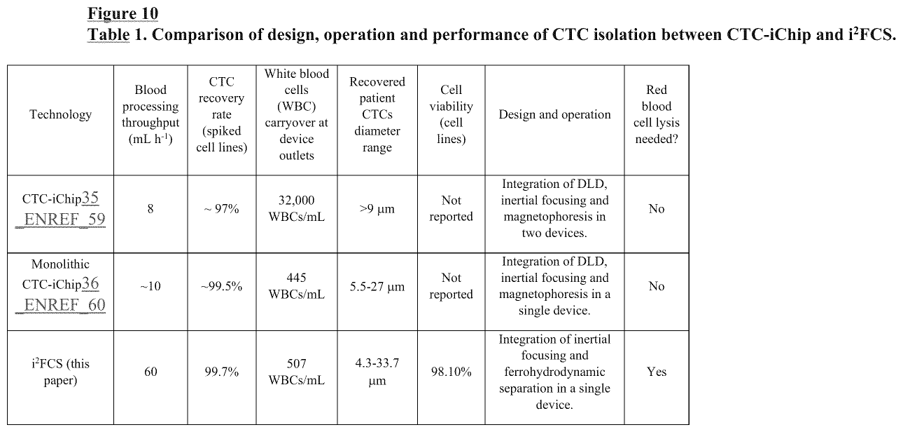

We also compared the performance of i²FCS to two generations of CTC-iChip in Table 1 (FIG. 10).35⋅ 36 i²FCS had six times higher blood sample flow rate (60 mL h⁻¹ for i²FCS versus 10 mL h⁻¹ for monolithic CTC-iChip). Both i²FCS and CTC-iChip depleted roughly the same amount of WBCs from blood samples (507 cells/mL carryover for i²FCS versus 445 cells/mL carryover for monolithic CTC-iChip). While the reported cancer cells recovery rates were almost the same for i²FCS and CTC-iChip using spiked cancer cells (99.7% for i²FCS versus 99.5% for monolithic CTC-iChip), the recovered CTCs from patient samples showed different physical diameter ranges, with i²FCS being able to isolate patient CTCs with a broader physical diameter range than CTC-iChip (4.3-33.7 μm for i²FCS versus 5.5-27 μm for monolithic CTC-iChip). i²FCS has an advantage of being able to recover small CTCs, because it does not differentiate CTCs and blood cells based on their physical diameters. Instead it uses the contrast of cellular magnetization for separation. This working principle ensured that all CTCs were separated regardless of their diameters. On the other hand, CTC-iChip integrated deterministic lateral displacement (DLD) to deplete red blood cells, inertial focusing to concentrate nucleated cells, and magnetophoresis to separate magnetically labeled CTCs. The size-based DLD stage in CTC-iChip could potentially remove small CTCs of similar size to red blood cells (6-8 μm). This slight selection bias might explain the diameter difference in recovered CTCs between the two methods. Finally, CTC-iChip could process whole blood without red-blood cell lysis while i²FCS methods performed red blood cell lysis on the sample prior to separation. Even though the cancer cell loss due to lysis step was demonstrated to be negligibly small (˜0.08%) in cancer cell line control experiments,32 it would be difficult to characterize such CTC loss in patient samples. In summary, i²FCS had the advantages of higher cell-processing throughput and sample flow rate, recovering CTCs with broader physical diameters, but lacked the ability to process whole blood when comparing to CTC-iChip.

### CONCLUSION

The present example demonstrates an integrated method that allowed for the first time simultaneous biochemical and functional phenotyping of patient-derived single circulating tumor cells. The method leveraged an integrated inertial ferrohydrodynamic cell separation (i²FCS) approach for a tumor cell marker and size agnostic isolation of CTCs from patient samples. This approach yielded remarkable CTC isolation performance including a complete isolation of CTCs from blood samples with a 99.70% recovery rate, an ultrahigh throughput of >600 millions of nucleated cells per hour, a ultrahigh blood processing flow rate of 60 mL h⁻¹, and an extremely low carryover of ˜507 WBCs for every one milliliter of blood processed. Furthermore, isolated CTCs from i²FCS preserved their functional properties and enabled their biochemical and functional phenotypes to be quantitively queried via a single cell migration assay.

In samples collected from two metastatic lung cancer patients, i²FCS and the migration assay enabled the sensitive profiling of CTCs' heterogeneity according to their surface antigen levels and migration phenotypes. CTCs profiled in samples collected from the patients revealed that there was a great level of diversity in the phenotypes of CTCs. CTCs exhibited variable levels of epithelial and mesenchymal antigen expressions and morphologies, confirming the marker and size agnostic isolation of the approach. Isolated cells were accessed for their motility towards a gradient of growth factors in a migration assay with single-cell resolution, revealing the existence of a high-motility subpopulation of CTCs in one of the patients' sample.

The i²FCS and migration assay approach could be potentially adapted to a variety of applications in cancer research. CTCs isolated from the i²FCS can readily be recovered with intactness and preserved biological functions, therefore facilitating further downstream analysis and culture. This approach allows multiplexed queries of functional CTCs, which makes it possible to analyze CTCs for their complex roles in metastasis. Experiments using this approach can be implemented using a standard syringe pump with microfluidic devices that are straightforward to fabricate and operate, making it relatively easy for laboratory adoptions.

### Materials and Methods

### Modeling and Simulation

Magnetic field and particle separation performance was simulated and optimized in MATLAB (MathWorks, Natick, MA) using a physical model, which predicted trajectories of cancer cells and labeled WBCs in the microfluidic channel coupled with a sextuple configuration of magnets.32 ⋅ 33 (incorporated herein by reference)

### Microfluidic Device Fabrication

The master mold containing the microfluidic structures was fabricated using standing photolithography methods with SU-8 2025 photoresist (Kayaku Advanced materials, Westborough, MA). The height of the structures was measured to be 60 μm. The 1 mm thick PDMS layer was prepared with Sylgard 184 silicone elastomer kit (Ellsworth Adhesives, Germantown, WI) in a 1:7 ratio of cross-linker and base, and cured at 60° C. for 4 hours. After bonding with the inlet and outlet layer (5 mm thick PDMS), the devices were oven baked at 80° C. for 20 minutes following by a hotplate at 150° C. for 1 hour. The device was placed within a custom aluminum manifold that held six N52 NdFeB permanent magnets (K&J Magnetics, Pipersville, PA) in a sextuple configuration. The magnets had a geometry of 50.8 mm×6.35 mm×6.35 mm (L×W×H) and had a remanent magnetization of 1.48 T each. Before each use, the devices were sterilized with 70% ethanol and then primed with 1×PBS supplemented with 0.5% (w/v) BSA and 2 mM EDTA (Thermo Fisher Scientific, Waltham, MA).

### Ferrofluid Synthesis and Characterization

The water-based ferrofluid was a colloidal suspension of maghemite nanoparticles, synthesized by a chemical co-precipitation method following developed protocol.58⋅ 59 (incorporated herein by reference). The saturation magnetization (1,107 A m⁻¹) and volume fraction of the ferrofluid (0.298%, v/v) were measured by a vibrating sample magnetometer (VSM, MicroSense, Lowell, MA). The viscosity of the ferrofluid (1.7 mPa s⁻¹) was characterized via a compact rheometer (Anton Paar, Ashland, VA) at room temperature. The diameter and morphology of maghemite nanoparticles were determined to be 10.91±4.87 nm (mean±s.d.) with a transmission electron microscopy (TEM; FEI, Eindhoven, the Netherlands).

### Cell Culture and Preparation

11 human cancer cell lines including four breast cancer cell lines (MCF7, MDA-MB-231, HCC1806, and HCC70), four non-small cell lung cancer (NSCLC) cell lines (A549, H1299, H3122, and H520), two small cell lung cancer (SCLC) cell lines (DMS79 and H59) and one prostate cancer cell line (PC-3) were purchased from ATCC (Manassas, VA). Cell cultures followed the manufacturing instructions. Breast cancer cell lines MCF7 and MDA-MB-231 were cultured in DMEM medium (Thermo Fisher Scientific, Waltham, MA) and the other cell lines were cultured in RPM 1640 medium (Thermo Fisher Scientific, Waltham, MA). DMEM and RPMI medium were supplemented with 10% (v/v) fetal bovine serum (FBS, Thermo Fisher Scientific, Waltham, MA), 1% (v/v) penicillin/streptomycin solution (Thermo Fisher Scientific, Waltham, MA), and 0.1 mM non-essential amino acid (NEAA, Thermo Fisher Scientific, Waltham, MA). All the cell lines were cultured at 37° C. with 5% CO₂. When the cells grown into 80% confluence, cells were washed twice with PBS by gently shaking the cell culture flask. This step was required to remove dead cells and debris. Cells were released with 0.05% trypsin-EDTA solution (Thermo Fisher Scientific, Waltham, MA), centrifugated (5 min, 500 g) to remove the supernatant, and resuspend in 1× Dulbecco's Phosphate Buffered Saline (DPBS, Thermo Fisher Scientific, Waltham, MA). To track the cell trajectories in the i²FCS device, cells were either stained with 3 μM CellTracker Green or 3 μM CellTracker Orange (Thermo Fisher Scientific, Waltham, MA) for 30 minutes at 37° C. and then washed and resuspended with culture medium. Cells were counted with Countess 2 (Thermo Fisher Scientific, Waltham, MA) and diluted to 10⁴ cells per mL with culture medium. After dilution, the exact number of cells was confirmed with Nageotte counting chamber (Hausser Scientific, Horsham, PA). Variable number (10, 50, 100, and 200) of cancer cells were spiked into 0.015% (v/v) ferrofluid for spiking experiments.

Recovery Rate and Purity Calculation of i²FCS

Cells collected from the CTC outlet and WBC outlet were stained with 2 μM DAPI (Thermo Fisher Scientific, Waltham, MA) to stain cell nucleus, and counted with a Nageotte counting chamber. Cells with CellTracker (Green/Orange) signal were identified as cancer cells, while other cells only expressing DAPI signal were identified as WBCs. The recovery rate of i²FCS was calculated by (N_{cancer_cell@CTC_outlet}/(N_{cancer_cell@CTC_outlet}+N_{cancer_cell@WBC_outlet}))×100%. The purity was characterized by the WBC carryover N_{WBC@CTC_outlet}, the depletion rate (1−N(N_{WBC@CTC_outlet}/N_{Total_WBC})×100%, and the log depletion rate log(N_{Total_WBC}/N_{WBC@CTC_outlet}).

### Cell Morphology Characterization

Cells suspended in PBS were deposited on a microscope slide and imaged with an inverted microscope (Axio Observer, Carl Zeiss, Germany) in bright field mode. Cell morphologies were analyzed with ImageJ software. Effective cell diameter was measured as the maximum Feret diameter of the cells under bright-field imaging.

### Cell Viability and Proliferation Characterization

Short-term cell viability of lung cancer cell line H1299 after i²FCS processing was characterized with a Live/Dead assay (Thermo Fisher Scientific, Waltham, MA) following the manufacturer's protocol. All cells are alive at the start of the viability characterization. Dead cells and cell debris were removed by PBS wash after cell culture. For long-term proliferation, the isolated H1299 cells from i²FCS device were washed three times with cell culture medium to remove the ferrofluid, and then the cells were re-suspended with culture medium and transferred into a T25 flask. (Corning, Corning, NY). The cells were then cultured at 37° C. (5% CO₂) under a humidified atmosphere. Cellular morphology was inspected every 24 hours.

### Human Sample Processing

Complete blood count (CBC) reports of cancer patients' blood samples were used to determine the number of WBCs to optimize WBC labeling. Whole blood was firstly labeled with biotinylated antibodies including anti-CD45 (eBioscience, San Diego, CA), anti-CD45RA (eBioscience, San Diego, CA), anti-16 (eBioscience, San Diego, CA), anti-66b (Biolegend, San Diego, CA), and anti-CD3 (Biolegend, San Diego, CA) for 30 minutes at room temperature. The antibody-conjugated blood was lysed with RBC lysis buffer (eBioscience, San Diego, CA) for 5 minutes following by centrifugation (500 g, 5 minutes) at room temperature. After removing the supernatant, the cells were resuspended with 1×PBS and incubated with washed Dynabeads (Thermo Fisher Scientific, Waltham, MA) for 30 minutes on a rocker. All the labeling and washing procedures were performed following the manufacturer's protocol. Blood cells were suspended in the same volume of 0.015% (v/v) ferrofluid supplemented with 0.1% (v/v) Pluronic F-68 surfactant (Thermo Fisher Scientific, Waltham, MA) before processing using the device.

### CTC Identification

After device processing, isolated cells were concentrated through centrifugation (600 g, 5 minutes) and immobilized onto poly-L-lysine (Sigma-Aldrich, St. Louis, Mo) coated glass slides. Isolated cells were fixed with 4% (w/v) paraformaldehyde (Santa Cruz Biotechnology, Dallas, TX) for 10 minutes and subsequently permeabilized with 0.1% (v/v) Triton X-100 (Alfa Aesar, Haverhill, MA) in PBS for 10 minutes at room temperature. Cells were then blocked with Ultracruz blocking reagent (Santa Cruz Biotechnology, Dallas, TX) for 30 minutes at room temperature to block nonspecific binding sites. Cells were then immunostained overnight at 4° C. with primary antibodies including EpCAM-Alexa Fluor 488, N-cadherin-Alex Fluor 594, Vimentin-Alex Fluor 647 (Santa Cruz Biotechnology, Dallas, TX), CD45-PE (BD Bioscience, San Jose, CA). Cells were stored in mounting medium supplemented with DAPI (Fluoroshield™ with DAPI, Sigma-Aldrich, St. Louis, Mo).

### Migration Assay of Isolated CTCs

Isolated CTCs were loaded into a microfluidic migration device for single cell migration assay.10% Fetal Bovine Serum (FBS, Thermo Fisher Scientific, Waltham, MA), 20 ng mL⁻¹ epidermal growth factor (EGF, Thermo Fisher Scientific, Waltham, MA), and 20 ng mL⁻¹ basic fibroblast growth factor (bFGF, Thermo Fisher Scientific, Waltham, MA) were used as the chemoattractants for the CTCs, while 5 μg mL⁻¹ Slit2 (Thermo Fisher Scientific, Waltham, MA) was used to inhibit the migration of WBCs. After cell loading, migration assay was performed in an incubator (37° C., 5% CO₂) for 24 hours. Cells was immunofluorescently stained in the device to identify their cell types.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

### REFERENCES

1. M. Poudineh, E. H. Sargent, K. Pantel and S. O. Kelley, Nat Biomed Eng, 2018, 2, 72-84.
2. C. Alix-Panabieres and K. Pantel, Nature Biomedical Engineering, 2017, 1.
3. J. Massague and A. C. Obenauf, Nature, 2016, 529, 298-306.
4. M. G. Krebs, R. L. Metcalf, L. Carter, G. Brady, F. H. Blackhall and C. Dive, Nat Rev Clin Oncol, 2014, 11, 129-144.
5. E. Heitzer, I. S. Hague, C. E. S. Roberts and M. R. Speicher, Nat Rev Genet, 2019, 20, 71-88.
6. N. Ma and S. S. Jeffrey, Science, 2020, 367, 1424-1425.
7. S. Riethdorf, H. Fritsche, V. Muller, T. Rau, C. Schindibeck, B. Rack, W. Janni, C. Coith, K. Beck, F. Janicke, S. Jackson, T. Gornet, M. Cristofanilli and K. Pantel, Clinical Cancer Research, 2007, 13, 920-928.
8. A. Romiti, S. Raffa, R. Di Rocco, M. Roberto, A. Milano, A. Zullo, L. Leone, D. Ranieri, F. Mazzetta, E. Medda, I. Sarcina, V. Barucca, C. D'Antonio, V. Durante, M. Ferri, M. R. Torrisi and P. Marchetti, J Gastrointest Liver, 2014, 23, 279-284.
9. F. C. Bidard, D. J. Peeters, T. Fehm, F. Nole, R. Gisbert-Criado, D. Mavroudis, S. Grisanti, D. Generali, J. A. Garcia-Saenz, J. Stebbing, C. Caldas, P. Gazzaniga, L. Manso, R. Zamarchi, A. F. de Lascoiti, L. De Mattos-Arruda, M. Ignatiadis, R. Lebofsky, S. J. van Laere, F. Meier-Stiegen, M. T. Sandri, J. Vidal-Martinez, E. Politaki, F. Consoli, A. Bottini, E. Diaz-Rubio, J. Krell, S. J. Dawson, C. Raimondi, A. Rutten, W. Janni, E. Munzone, V. Caranana, S. A. Agelaki, C. Almici, L. Dirix, E. F. Solomayer, L. Zorzino, H. Johannes, J. S. Reis, K. Pantel, J. Y. Pierga and S. Michiels, Lancet Oncol, 2014, 15, 406-414.
10. A. W. Lambert, D. R. Pattabiraman and R. A. Weinberg, Cell, 2017, 168, 670-691.
11. C. L. Chaffer and R. A. Weinberg, Science, 2011, 331, 1559-1564.
12. M. Poudineh, P. Aldridge, S. Ahmed, B. J. Green, L. Kermanshah, V. Nguyen, C. Tu, R. M. Mohamadi, R. K. Nam, A. Hansen, S. S. Sridhar, A. Finelli, N. E. Fleshner, A. M. Joshua, E. H. Sargent and S. O. Kelley, Nat Nanotechnol, 2017, 12, 274-+.
13. Y. H. Cheng, Y. C. Chen, E. Lin, R. Brien, S. Jung, Y. T. Chen, W. Lee, Z. Hao, S. Sahoo, H. Min Kang, J. Cong, M. Burness, S. Nagrath, S. W. M and E. Yoon, Nat Commun, 2019, 2163.
14. E. Sahai, Nat Rev Cancer, 2007, 7, 737-749.
15. L. Keller and K. Pantel, Nat Rev Cancer, 2019, 19, 553-567.
16. B. Strilic and S. Offermanns, Cancer Cell, 2017, 32, 282-293.
17. P. S. Steeg, Nature reviews cancer, 2016, 16, 201-218.
18. P. Friedl and K. Wolf, Nat Rev Cancer, 2003, 3, 362-374.
19. C. D. Paul, P. Mistriotis and K. Konstantopoulos, Nat Rev Cancer, 2017, 17, 131-140.
20. C. Alix-Panabieres and K. Pantel, Nat Rev Cancer, 2014, 14, 623-631.
21. M. Poudineh, E. H. Sargent, K. Pantel and S. O. Kelley, Nat Biomed Eng, 2018, 2, 72-84.
22. C. Alix-Panabieres, K. Bartkowiak and K. Pantel, Mol Oncol, 2016, 10, 443-449.
23. Y. Zhang, W. Zhang and L. Qin, Angew Chem Int Ed Engl, 2014, 53, 2344-2348.
24. C. L. Yankaskas, K. N. Thompson, C. D. Paul, M. I. Vitolo, P. Mistriotis, A. Mahendra, V. K. Bajpai, D. J. Shea, K. M. Manto, A. C. Chai, N. Varadarajan, A. Kontrogianni-Konstantopoulos, S. S. Martin and K. Konstantopoulos, Nat Biomed Eng, 2019, 3, 452-465.
25. Y. C. Chen, S. G. Allen, P. N. Ingram, R. Buckanovich, S. D. Merajver and E. Yoon, Sci Rep, 2015, 5, 9980.
26. I. Y. Wong, S. Javaid, E. A. Wong, S. Perk, D. A. Haber, M. Toner and D. Irimia, Nat Mater, 2014, 13, 1063-1071.
27. M. Poudineh, M. Labib, S. Ahmed, L. N. Nguyen, L. Kermanshah, R. M. Mohamadi, E. H. Sargent and S. O. Kelley, Angew Chem Int Ed Engl, 2017, 56, 163-168.
28. D. Di Carlo, D. Irimia, R. G. Tompkins and M. Toner, Proc Natl Acad Sci USA, 2007, 104, 18892-18897.
29. H. Amini, W. Lee and D. Di Carlo, Lab Chip, 2014, 14, 2739-2761.
30. D. Di Carlo, Lab Chip, 2009, 9, 3038-3046.
31. R. E. Rosensweig, Ferrohydrodynamics, Cambridge University Press, Cambridge, 1985.
32. W. Zhao, Y. Liu, B. D. Jenkins, R. Cheng, B. N. Harris, W. Zhang, J. Xie, J. R. Murrow, J. Hodgson, M. Egan, A. Bankey, P. G. Nikolinakos, H. Y. Ali, K. Meichner, L. A. Newman, M. B. Davis and L. Mao, Lab Chip, 2019, 19, 1860-1876.
33. Y. Liu, W. Zhao, R. Cheng, B. N. Harris, J. R. Murrow, J. Hodgson, M. Egan, A. Bankey, P. G. Nikolinakos, T. Laver, K. Meichner and L. Mao, Lab Chip, 2021, DOI:
34. A. Mishra, T. D. Dubash, J. F. Edd, M. K. Jewett, S. G. Garre, N. M. Karabacak, D. C. Rabe, B. R. Mutlu, J. R. Walsh and R. Kapur, Proceedings of the National Academy of Sciences, 2020, 117, 16839-16847.
35. E. Ozkumur, A. M. Shah, J. C. Ciciliano, B. L. Emmink, D. T. Miyamoto, E. Brachtel, M. Yu, P. I. Chen, B. Morgan, J. Trautwein, A. Kimura, S. Sengupta, S. L. Stott, N. M. Karabacak, T. A. Barber, J. R. Walsh, K. Smith, P. S. Spuhler, J. P. Sullivan, R. J. Lee, D. T. Ting, X. Luo, A. T. Shaw, A. Bardia, L. V. Sequist, D. N. Louis, S. Maheswaran, R. Kapur, D. A. Haber and M. Toner, Science Translational Medicine, 2013, 5, 179ra147.
36. F. Fachin, P. Spuhler, J. M. Martel-Foley, J. F. Edd, T. A. Barber, J. Walsh, M. Karabacak, V. Pai, M. Yu, K. Smith, H. Hwang, J. Yang, S. Shah, R. Yarmush, L. V. Sequist, S. L. Stott, S. Maheswaran, D. A. Haber, R. Kapur and M. Toner, Sci Rep, 2017, 7, 10936.
37. M. A. Nieto, R. Y. Huang, R. A. Jackson and J. P. Thiery, Cell, 2016, 166, 21-45.
38. J. P. Thiery, H. Acloque, R. Y. Huang and M. A. Nieto, Cell, 2009, 139, 871-890.
39. J. P. Thiery, Nat Rev Cancer, 2002, 2, 442-454.
40. Y. Liu, W. Zhao, R. Cheng, A. Puig, J. Hodgson, M. Egan, C. N. Cooper Pope, P. G. Nikolinakos and L. Mao, Lab Chip, 2021, 21, 2738-2750.
41. J. J. Nieva and P. Kuhn, Future Oncology, 2012, 8, 989-998.
42. D. J. Peeters, G. G. Van den Eynden, P. J. van Dam, A. Prove, I. H. Benoy, P. A. van Dam, P. B. Vermeulen, P. Pauwels, M. Peeters, S. J. Van Laere and L. Y. Dirix, Br J Cancer, 2011, 104, 1472-1477.
43. R. A. Harouaka, M. Nisic and S. Y. Zheng, J Lab Autom, 2013, 18, 455-468.
44. D. Marrinucci, K. Bethel, D. Lazar, J. Fisher, E. Huynh, P. Clark, R. Bruce, J. Nieva and P. Kuhn, J Oncol, 2010, 2010, 861341.
45. M. Yu, A. Bardia, B. Wittner, S. L. Stott, M. E. Smas, D. T. Ting, S. J. Isakoff, J. C. Ciciliano, M. N. Wells, A. M. Shah, K. F. Concannon, M. C. Donaldson, L. V. Sequist, E. Brachtel, D. Sgroi, J. Baselga, S. Ramaswamy, M. Toner, D. A. Haber and S. Maheswaran, Science, 2013, 339, 580-584.
46. S. L. Stott, C.-H. Hsu, D. I. Tsukrov, M. Yu, D. T. Miyamoto, B. A. Waltman, S. M. Rothenberg, A. M. Shah, M. E. Smas, G. K. Korir, F. P. Floyd, A. J. Gilman, J. B. Lord, D. Winokur, S. Springer, D. Irimia, S. Nagrath, L. V. Sequist, R. J. Lee, K. J. Isselbacher, S. Maheswaran, D. A. Haber and M. Toner, Proceedings of the National Academy of Sciences, 2010, 107, 18392-18397.
47. R. J. Petrie, A. D. Doyle and K. M. Yamada, Nat Rev Mol Cell Biol, 2009, 10, 538-549.
48. E. T. Roussos, M. Balsamo, S. K. Alford, J. B. Wyckoff, B. Gligorijevic, Y. Wang, M. Pozzuto, R. Stobezki, S. Goswami, J. E. Segall, D. A. Lauffenburger, A. R. Bresnick, F. B. Gertler and J. S. Condeelis, J Cell Sci, 2011, 124, 2120-2131.
49. E. T. Roussos, J. S. Condeelis and A. Patsialou, Nat Rev Cancer, 2011, 11, 573-587.
50. J. Y. Wu, L. Feng, H. T. Park, N. Havlioglu, L. Wen, H. Tang, K. B. Bacon, Z. Jiang, X. Zhang and Y. Rao, Nature, 2001, 410, 948-952.
51. L. Boneschansker, J. Yan, E. Wong, D. M. Briscoe and D. Irimia, Nat Commun, 2014, 5, 4787.
52. K. Wolf, S. Alexander, V. Schacht, L. M. Coussens, U. H. von Andrian, J. van Rheenen, E. Deryugina and P. Friedl, Semin Cell Dev Biol, 2009, 20, 931-941.
53. B. Weigelin, G. J. Bakker and P. Friedl, Intravital, 2012, 1, 32-43.
54. P. C. Benias, R. G. Wells, B. Sackey-Aboagye, H. Klavan, J. Reidy, D. Buonocore, M. Miranda, S. Kornacki, M. Wayne, D. L. Carr-Locke and N. D. Theise, Sci Rep, 2018, 8, 4947.
55. E. Sahai, Curr Opin Genet Dev, 2005, 15, 87-96.
56. K. Wolf, I. Mazo, H. Leung, K. Engelke, U. H. von Andrian, E. I. Deryugina, A. Y. Strongin, E. B. Brocker and P. Friedl, J Cell Biol, 2003, 160, 267-277.
57. E. Sahai and C. J. Marshall, Nat Cell Biol, 2003, 5, 711-719.
58. W. J. Zhao, R. Cheng, S. H. Lim, J. R. Miller, W. Z. Zhang, W. Tang, J. Xie and L. D. Mao, Lab on a Chip, 2017, 17, 2243-2255.
59. Y. Liu, W. Zhao, R. Cheng, M. Logun, M. D. M. Zayas-Viera, L. Karumbaiah and L. Mao, Lab Chip, 2020, 20, 3187-3201.

We claim:

1. A method of enriching target rare cells in a biological sample, the method comprising:
combining the biological sample with a plurality of magnetic microbeads adapted to specifically conjugate with white blood cells (WBCs) such that a majority of WBCs in the sample are conjugated to one or more magnetic microbeads to produce a magnetically labeled biological sample;
combining the magnetically labeled biological sample with a colloidally stable ferrofluid to produce a mixed ferrofluid biological sample;
flowing the mixed ferrofluid biological sample through an inertial focusing stage comprising two or more sigmoidal microchannels with a plurality of alternating curvatures, such that rare cells and WBCs in the mixed ferrofluid biological sample are focused into two or more narrow focused fluid sample streams;
flowing the two or more focused fluid sample streams through a ferrohydrodynamic separation stage comprising a ferrohydrodynamic separation channel and a magnetic source configured to produce a substantially symmetric magnetic field having a field maximum along an inner longitudinal axis of the ferrohydrodynamic separation channel sufficient to cause the white blood cells conjugated to the magnetic beads flowing into the ferrohydrodynamic separation channel to be focused toward a central longitudinal axis of the ferrohydrodynamic separation channel and to cause target rare cells in the sample to be deflected towards an outer portion of the ferrohydrodynamic separation channel;
separating the magnetic-bead-conjugated WBCs flowing along a central longitudinal axis of the ferrohydrodynamic separation channel from target rare cells flowing along an outer portion of the ferrohydrodynamic separation channel to produce an enriched biological sample.

2. The method of claim 1, wherein the rare cells are circulating tumor cells (CTCs) and the biological sample is a red blood cell-lysed blood sample from a patient.

3. The method of claim 1, wherein the flow rate of the mixed ferrofluid biological sample is about 500-2000 μl/min.

4. The method of claim 1, wherein the concentration of the ferrofluid is about 0.005-0.05%.

5. The method of claim 1, wherein over 95% of WBC's are separated from the biological sample to produce the enriched biological sample.

6. The method of claim 1, wherein over 99% of WBC's are separated from the biological sample to produce the enriched biological sample.

7. The method of claim 1, wherein the magnetic source comprises an array of magnets comprising a first array and second array, wherein the ferrohydrodynamic separation stage is sandwiched between and substantially centrally aligned between the first magnet array and the second magnet array, wherein the magnets in the first array are oriented to repel the magnets in the second array.

8. The method of claim 7, wherein the array of magnets comprises six magnets arranged in a sextuple configuration.

9. The method of claim 1, further comprising introducing the enriched biological sample into a microfluidic chemotactic cell migration unit and sorting the target rare cells into different migratory phenotypes based on at least one of a speed and distance of migration of target rare cells with respect to one or more chemo-modulatory compounds flowing in a portion of the microfluidic chemotactic cell migration unit.

10. The method of claim 9, wherein the target rare cells are circulating tumor cells (CTCs) and the one or more chemo-modulatory compounds comprises at least one compound that is a chemoattractant for CTCs having an invasive phenotype.

11. The method of claim 10, wherein the chemoattractant for CTCs is selected from the group consisting of Fetal Bovine Serum (FBS), epidermal growth factor (EGF), and basic fibroblast growth factor (bFGF).

12. The method of claim 10, wherein the rare cells are target circulating tumor cells (CTCs) and the one or more chemo-modulatory compounds comprises at least one compound that is a migration inhibitor for WBCs.

13. The method of claim 12 wherein the at least one migration inhibitor compound for WBCs is Slit2.

14. The method of claim 8, wherein the enriched biological sample is incubated in microfluidic chemotactic cell migration unit with the chemo-modulatory compounds for about 3 to 48 hours.

15. The method of claim 14 wherein a gradient of chemo-modulatory compounds is maintained by continuous perfusion during the incubation time.

16. The method of claim 1, wherein conjugating the WBCs in the sample to magnetic microbeads comprises:
combining the WBC with two or more WBC biomarker antibodies and then conjugating the biomarker antibodies to the magnetic microbeads.

17. The method of claim 16, wherein the biomarker antibodies are biotinylated and the magnetic microbeads are coated with streptavidin, and wherein the two or more WBC biomarker antibodies are selected from the group consisting of: CD45, CD45RA, CD66b, CD16, and CD3.

18. An integrated inertial ferrohydrodynamic cell separation (i²FCS) microfluidic unit comprising:
a sample inlet configured to receive a mixed ferrofluid biological sample comprising a biocompatible ferrofluid combined with a biological sample comprising target rare cells and magnetically labeled white blood cells (WBCs);
a filter section fluidly connected to the sample inlet, the filter section comprising a first microfluidic channel and one or more filters configured to remove a first plurality of waste particles from the mixed ferrofluid biological sample;
an inertial focusing stage fluidly connected to the filter section, wherein the first microfluidic channel splits into two or more sigmoidal microchannels with a plurality of alternating micro-curves, each sigmoidal microchannel configured to focus cells within the sample into a narrow stream to produce a focused fluid sample stream;
a ferrohydrodynamic separation stage fluidly connected to the inertial focusing stage such that the focused fluid sample streams from the two or more sigmoidal microchannels flow into a ferrohydrodynamic separation channel;
a waste outlet fluidly connected to and axially aligned with the ferrohydrodynamic separation channel to receive materials flowing through a central portion of the channel,
one or more target cell outlets each fluidly connected to the ferrohydrodynamic separation channel and offset from the center of the channel and configured to receive material flowing near the outer portion of the channel; and
one or more magnetic sources adjacent to ferrohydrodynamic separation stage and configured to produce a substantially symmetric magnetic field having a field maximum along a length of the ferrohydrodynamic separation channel sufficient to cause the white blood cells conjugated to the magnetic beads in the ferrohydrodynamic separation channel to be focused toward a center of the channel and to exit the channel via the waste outlet and to cause target rare cells to be deflected towards an outer portion of the ferrohydrodynamic separation channel and to exit the channel via the one or more target cell outlets.

19. A combined integrated inertial ferrohydrodynamic cell separation (i²FCS) and cell migration device comprising:
the i²FCS unit of claim 18 and a chemotactic cell migration unit comprising:
an enriched sample inlet to receive an enriched biological sample from the i²FCS unit;
a chemo-modulatory fluid inlet to receive a chemo-modulatory fluid comprising one or more chemo-modulatory compounds;
one or more sample flow channels fluidly connected to the enriched sample inlet and configured to flow the enriched biological sample;
one or more chemo-modulatory channels fluidly connected to the chemo-modulatory fluid inlet, configured to flow the chemo-modulatory fluid, and oriented substantially parallel to the one or more sample flow channels;
a plurality of migration microchannels connecting at least one sample flow channel to at least one chemo-modulatory channels, each of the plurality of migration microchannels oriented substantially perpendicular to the sample flow channel and the chemo-modulatory channel and in fluidic communication with both the sample flow channel and the chemo-modulatory channel, wherein the height of each migration microchannel is smaller than the height of each of the sample flow channel and the chemo-modulatory channels; and
at least one migratory target cell outlet at and end of a chemo-modulatory channel opposite the chemo-modulatory fluid inlet configured to collect migratory target cells that have migrated from the enriched sample inlet.
